(19) United States
(12) Patent Application Publication
(10) Pub. No.: US 2004/0170653 A1
(43) Pub. Date: Sep. 2, 2004

(54) Tumour peptide antigen produced from human mdm2 proto-oncogene

(76) Inventors: Thomas Stanislawski, Jugenheim (DE); Matthias Theobald, Mainz-Kastel (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/469,555

(22) PCT Filed: Mar. 1, 2002
(86) PCT No.: PCT/EP02/02250

(30) Foreign Application Priority Data
Mar. 1, 2001 (DE) 101 09 813.8

(51) Int. Cl. C07H 21/02; A61K 39/00; C07K 14/47

(52) U.S. Cl. 424/277.1; 514/44; 530/350; 536/23.1

(57) ABSTRACT

The invention relates to a universal tumour-associated oligopeptide, which is recognised by CD8-positive cytotoxic T-lymphocytes (CTL) as a peptide antigen and which causes a CTL-induced lysis and/or apoptosis of tumour or leukaemia cells. The oligopeptide has the amino acid sequence LLGDLFGV, which corresponds to the amino acid positions 81 to 88 of the hdm2 proto-oncoprotein, or an amino acid sequence that can be derived from said sequence, which constitutes the functional equivalent of the amino acid sequence LLGDLFGV. Said oligopeptide constitutes an epitope for CD8-positive CTLs and is suitable for inducing a restricted immune response of CD8-positive CTLs to the human leukocyte antigen of the molecular group MHC class I, allelomorph variant A2, against tumour and leukaemia cells.

The invention relates to a universal tumor-associated oligopeptide which is recognized as peptide antigen by CD8-positive cytotoxic T lymphocytes (CTL) and which produces a CTL-induced lysis and/or apoptosis of tumor or leukemia cells.

CD8-positive CTL are effector cells of the cellular immune system. Their function consists in the specific elimination of infected or degenerate endogenous cells. The CTL recognize, inter alia, tumor-specific or tumor-associated peptide antigens which are bound to major histocompatibility complex (MHC) molecules of class I and are presented on the surface of the degenerate cells. The recognition of the peptide antigens in the context of MHC class I molecules is carried out by specific membranous T-cell receptors (TCR) of the CTL. After recognition, the cell concerned is destroyed by the CTL lyzing the target cells and/or inducing the programmed cell death (apoptosis) of these target cells or releasing cytokines.

The recognition of target cells by CTL is facilitated by the expression of the CD8 coreceptor on CTL. The CD8 coreceptor binds to conserved regions of the α2 and α3 domains of the MHC class I molecule and thus contributes to the stabilization of the TCR-peptide-MHC complex.

Among the tumor-associated peptide antigens (TAA) which are presented on the surface of tumor cells in the context of MHC class I molecules, the “universal” TAA are of particular interest. “Universal” TAA are derived mainly from the cellular proteins, which are weakly expressed in normal cells and overexpressed in tumor cells. These proteins include, inter alia, the human homolog of the “mouse double minute 2” proto-oncogene (mdm2), the “human mdm2” or in short “hdm2” proto-oncoprotein, which is overexpressed not only in a number of solid tumors, but also in the hematological neoplasias (malignant hematological systemic disorders) AML, ALL and CLL. The oligopeptides resulting from the cellular processing of the hdm2 protein can be presented on the cell surface in the context of MHC class I molecules of the allele variant A2, subtype A2.1 (in short: A2.1; the most frequent MHC class I allele in the Caucasian population), and represent attractive target structures for CD8-positive CTL. The expression of hdm2 in normal tissues has not been intensively investigated until now. For mdm2 of the mouse, however, an increased expression of mdm2 mRNA in the testis and a lower expression in the thymus, ovary and the central nervous system, and an increased expression of mdm2 protein in the uterus has been detected.

A prerequisite for the development of immunotherapeutic procedures for the treatment of malignant oncoses is the identification of immunogenic tumor antigens. Such tumor antigens can be employed under certain conditions as a vaccine for the induction of T cells in general and of tumor-reactive T cells in particular with the aim that these T cells produce the remission and eradication of a certain tumor. In the case of melanomas, some peptide antigens are already known which are used in this manner for immunotherapy within clinical trials.

The present invention is based on the object of making available “universal” tumor-associated peptide antigens (universal TAA) which are recognized by CD8-positive CTL and produce a CTL-induced lysis and/or apoptosis of tumor or leukemia cells.

A solution to this object consists in the making available of an oligopeptide, which has (a) the amino acid sequence LLGDLFGV, which corresponds to the amino acid positions 81 to 88 of the hdm2 proto-oncoprotein, or which has an amino acid sequence derivable by amino acid substitution, deletion, insertion, addition, inversion and/or by chemical or physical modification of one or more amino acids thereof, which is a functional equivalent to the amino acid sequence LLGDLFGV, which (b) is an epitope for CD8-positive CTL, and which (c) is suitable for inducing an immune response restricted to human leukocyte antigen of the molecular group “MHC class I”, allele variant A2 (in short: A2) of CD8-positive CTL to tumor and leukemia cells.

An equivalent solution consists in the making available of a retro-inverse peptide or pseudopeptide analogous to this oligopeptide according to the invention, which instead of the —CO—NH— peptide bonds has nonpeptide bonds such as, for example, —NH—CO— bonds (Meziere et al. 1997).

Using the oligopeptide “hdm2 81-88”, a peptide antigen is for the first time made available whose amino acid sequence originates from the hdm2 oncoprotein. The hdm2 81-88 oligopeptide and its derivatives are ubiquitous, quantitatively tumor-associated CTL epitopes and thus yield the molecular basis for an hdm2-specific immunotherapy of malignant diseases.

The oligopeptides according to the invention (hdm2 81-88 and its derivatives) can be used in the active and passive immunization of patients having malignant solid oncoses and/or lymphohematopoietic neoplasias, in which the hdm2 epitope 81-88 is presented in the context of A2.1, in order to produce the induction, generation and expansion of hdm2 81-88-specific cytotoxic T lymphocytes, which are able specifically to destroy the tumor or leukemia cells of the patients concerned and thereby to bring about a cure.

In the course of the present invention, it has surprisingly been found that hdm2 is overexpressed in malignant hematological diseases also in the form of a multiple myeloma (or plasmocytoma), of a histiocytic lymphoma and of a CML-myeloblastic crisis, while it is not detectable in resting B cells, T cells, mononuclear cells of the peripheral blood, lung fibroblasts and physiologically activated dendritic and T cells. For the oligopeptide hdm2 81-88 and its derivatives, the advantage results from this of a broad indication area with negligibly low risk of an undesired attack on normal cells.

The derivatives of the hdm2 81-88 oligopeptide, compared with the oligopeptide itself, have the advantage that a potential functional self-tolerance (compared with the hdm2 81-88 oligopeptide) can be circumvented therewith at the T-cell level. While the hdm2 81-88 oligopeptide is under certain circumstances a “tolerogen” in the organism concerned (patient's body) on account of the (low) expression in some normal tissues, and is not immunogenic for the organism's own (patient's own) CTL, the derivatives of the hdm2 81-88 oligopeptide are as a rule recognized as antigens and induce the activation and expansion of CTL. These derivative-induced CTL as a rule have a high cross-reactivity to the hdm2 81-88 wild-type sequence and as a result also induce the lysis and/or apoptosis of those (tumor) cells which present hdm2 81-88 (in the context of A2, in particular of A2.1) on their surface. Particularly preferred derivatives of the hdm2 81-88 oligopeptide are those which occur naturally in other mammals or in vertebrates, e.g. hdm2 81-88 homologs of the mouse. The hdm2 (protein) homologs and the nucleic acids coding therefor can be obtained from the respective organism relatively easily, namely directly and using familiar isolation processes.

The oligopeptide hdm2 81-88 and its derivatives can be prepared by means of customary peptide synthesis processes, and the nucleotide sequences coding for these oligopeptides can be obtained using known chemical or using molecular biological processes.

The oligopeptides according to the invention (hdm2 81-88 and its derivatives) are suitable both for the in-vivo induction of T lymphocytes in the patient and for the in-vitro induction and expansion of appropriately reactive patient's own or patient-foreign T lymphocytes.

For an in-vivo induction and expansion of T lymphocytes in the patient various processes are possible, for example (a) the injection of the hdm2 81-88 oligopeptide and/or one or more of its derivatives as pure peptide or together with adjuvants or with cytokines or in a suitable release systems such as, for example, liposomes, (b) the injection of one or more nucleic acids coding at least for the hdm2 81-88 oligopeptide or for its derivatives—in “naked” or complexed form or in the form of viral or nonviral vectors or together with release systems such as cationic lipids or cationic polymers, (c) the loading of cells of autologous, allogenic, xenogenic or microbiological origin with the hdm2 81-88 oligopeptide or its derivatives or retro-inverse peptides or pseudopeptides analogous thereto, (d) the loading of cells of autologous, allogenic, xenogenic or microbiological origin with the hdm2 protein or homologs of other species, so that as a result the hdm2 81-88 oligopeptide or its derivatives is presented on the respective cells, or (●) the transfection or infection of cells of auto-logous, allogenic, xenogenic or microbiological origin with the nucleic acids coding at least for the peptide or its derivatives (again either in “naked” or complexed form or in the form of viral or nonviral vectors).

In the case of an in-vitro induction and expansion, the T lymphocytes obtained in-vitro are then administered to the patient by infusion or injection or like procedures.

The invention therefore also relates to the use of the hdm2 81-88 oligopeptide and/or its derivatives and/or retro-inverse peptides or pseudopeptides analogous thereto and/or at least one polynucleotide, which codes at least for the oligopeptide or its derivatives, for the production of diagnostics—in particular MHC tetramers or other structures, to which at least one such oligopeptide or retro-inverse peptide or pseudopeptide according to the invention is associated —and/or prophylactics and/or therapeutics (in particular vaccines) for the detection and/or the influencing and/or generation and/or expansion and/or control of the activation and functional state of T cells, in particular CD8-positive CTL.

Possible therapeutics and/or prophylactics are in particular vaccines or injections or infusion solutions, which as active compound (a) contain the hdm2 81-88 oligopeptide and/or at least one derivative thereof and/or at least one retro-inverse peptide or pseudopeptide analogous to this oligopeptide or to its derivative, and/or which contain (b) a nucleic acid, which codes at least for the hdm2 81-88 oligopeptide or at least for one of its derivatives, and/or which contain (c) T lymphocytes produced in-vitro, which are directed specifically against the hdm2 81-88 oligopeptide and/or its derivatives and/or against a retro-inverse peptide or pseudopeptide analogous to this oligopeptide or to its derivative(s).

For the preparation of the diagnostics or alternatively of the therapeutics or alternatively of the prophylactics, recombinant DNA or RNA vector molecules, which contain one or more polynucleotide(s), are in particular also suitable which code for at least the hdm2 81-88 oligopeptide and/or for at least one derivative thereof, and which are transcribable or expressible in cells of autologous, allogenic, xenogenic or microbiological origin. The invention therefore also comprises those recombinant DNA or RNA vector molecules and host cells, which contain these vector molecules.

As a diagnostic or therapeutic or prophylactic or generally for a detection and/or manipulation of hdm2 overexpressing cells, according to the invention polyclonal, monoclonal or recombinant antibodies can also be employed which are directed against the hdm2 81-88 oligopeptide and/or against its derivative(s) and/or against a retro-inverse peptide or pseudopeptide analogous to the oligopeptide or its derivative or which react with a complex of the oligopeptide(s) concerned or its derivative(s) or peptide(s) and/or pseudopeptide(s) retro-inverse thereto and HLA-A2. The use of the hdm2 81-88 oligopeptide and/or its derivative(s) and/or a retro-inverse peptide or pseudopeptide analogous to the oligopeptide or one of its derivatives for the preparation of polyclonal, monoclonal or recombinant antibodies against such an oligopeptide or retro-inverse peptide or pseudopeptide according to the invention and the antibody(ies) concerned per se are consequently likewise part of the present invention.

As a diagnostic or therapeutic or prophylactic or generally for a detection and/or manipulation of hdm2 overexpressing cells, according to the invention polyclonal, monoclonal or recombinant A2-restricted T-cell receptors or molecules functionally equivalent thereto can also be employed, which are specific for the hdm2 81-88 oligopeptide and/or its derivatives and/or for retro-inverse peptides or pseudopeptides analogous thereto. The T-cell receptors or molecules functionally equivalent thereto can be of autologous, allogenic or xenogenic origin.

The subject matter of the present invention consequently primarily also includes:

the use of the hdm2 81-88 oligopeptide and/or its derivatives and/or retro-inverse peptides or pseudopeptides analogous thereto or the use of polynucleotides having a nucleotide sequence which codes at least for the hdm2 81-88 oligopeptide and/or a derivative thereof for the preparation of polyclonal, monoclonal or recombinant A2-restricted T-cell receptors or molecules functionally equivalent thereto having specificity for such an oligopeptide or retro-inverse peptide or pseudopeptide according to the invention,

the T-cell receptor(s) concerned per se and molecules functionally equivalent thereto,

and polynucleotides which code for these T-cell receptors or molecules functionally equivalent thereto,

expression vectors having the ability for the expression of these T-cell receptors or molecules functionally equivalent thereto.

The invention moreover comprises reagents for the in-vivo or in-vitro activation of T cells, in particular CD8-positive CTL, which are characterized in that they are prepared using the hdm2 81-88 oligopeptide and/or at least one of its derivatives and/or at least one retro-inverse peptide or pseudopeptide analogous thereto and/or using at least one polynucleotide which codes at least for the oligopeptide or its derivative(s) and/or using the hdm2 protein or homologs thereto of other species. These reagents can in particular be therapeutics, especially vaccines.

The invention is illustrated in greater detail with figures below with the aid of preparation and use examples. The abbreviations used are:

| | |
|---|---|
| A2 | human leukocyte antigen of the molecular |
| | group “MHC class I”, allele variant “A2” |
| A2.1 | human leukocyte antigen of the molecular |
| | group “MHC class I”, allele variant |
| | “A2”, subtype “A2.1” |
| A2K^{b} | A2.1/K^{b} = MHC class I molecule from α₁ |
| | and α₂ domains of A2 and α₃ domain of K^{b} |
| ALL | acute lymphatic leukemia |
| AML | acute myeloid leukemia |
| APS | ammonium persulfate |
| APC | antigen-presenting cell |
| ATCC | American Type Culture Collection |
| ATP | adenosine-5′-triphosphate |
| B-ALL | B-cell ALL |
| bkgd | nonspecific fluorescence intensity |
| bp | base pair |
| BSA | bovine serum albumin |
| C-terminal | carboxyl-terminal |
| CD | differentiation cluster |
| CD8 | human CD8 α/β-coreceptor |
| CDR | complementarity-determining region |
| CLL | chronic lymphatic leukemia |
| CML | chronic myeloid leukemia |
| CMV | cytomegalovirus |
| Con A | concanavalin A |
| DMSO | dimethyl sulfoxide |
| DNA | deoxyribonucleic acid |
| DSMZ | German collection of microorganisms and |
| | cell cultures |
| DTT | dithiothreitol |
| DC | dendritic cell |
| E:T | effector to target cell ratio |
| EBV | Epstein-Barr virus |
| EDTA | ethylenediamine tetraacetate |
| ER | endoplasmic reticulum |
| FACS | fluorescence-activated cell sorter |
| FCS | fetal calf serum |
| FITC | fluorescein isothiocyanate |
| Flu M1 | A/PR/8/34 influenza virus matrix protein |
| | M1 |
| G-418 | geneticin (neomycin antibiotic) |
| GM-CSF | granulocyte-macrophage colony stimulating |
| | factor |
| HBV pol | hepatitis B virus polymerase |
| hdm2 | human homolog of mdm2 |
| HEPES | N-(2-hydroxyethyl)piperizane-N′-ethane- |
| | sulfonic acid |
| HLA | human leukocyte antigen |
| HLA-A2.1 | human leukocyte antigen of the molecular |
| | group “MHC class I”, allele variant |
| | “A2”, subtype “A2.1” |
| HPLC | high-pressure liquid chromatography |
| IFA | incomplete Freund's adjuvant |
| IFN | interferon |
| Ig | immune globulin |
| IL | interleukin |
| kb | kilobase pair |
| K^{b} | H-2K^{b} |
| kDa | kilodalton |
| LB | Luria-Bertani |
| LCL | lymphoblastoid cell line |
| LMP | low molecular mass polypeptide |
| LPS | lipopolysaccharide |
| mdm2 | mouse double minute 2 |
| MHC | major histocompatibility complex |
| Mio | million |
| mut | mutated |
| N-terminal | amino-terminal |
| OD | optical density |
| PBMC | mononuclear cells of the peripheral |
| | blood |
| PBS | phosphate-buffered saline solution |
| PG-E₂ | prostaglandin E₂ |
| PHA | phytohemagglutinin |
| PMSF | phenylmethylsulfonyl fluoride |
| PVDF | polyvinylidene difluoride |
| Rad | radiation absorbed dose |
| RP | reverse phase |
| SDS | sodium dodecylsulfate |
| SDS-PAGE | SDS-polyacrylamide gel electrophoresis |
| SL | specific lysis |
| SV-40 | Simian virus 40 |
| TAA | tumor-associated antigen(s) |
| TAP | transporter associated with antigen |
| | processing |
| TBE | tris-boric acid-EDTA |
| TE | tris-EDTA |
| TEMED | N,N,N′,N′-tetramethylethylenediamine |
| TFA | trifluoroacetic acid |
| TIL | tumor-infiltrating lymphocytes |
| TNF-α | tumor necrosis factor-α |
| Tris | tris(hydroxymethyl)aminomethane |
| TCR | T-cell receptor |
| u | international units |
| rpm | revolutions per minute |
| VSV-N | vesicular stomatitis virus nucleoprotein |
| v/v | volume per volume |
| wt | wild-type |
| w/v | mass per volume |
| CTL | cytotoxic T lymphocytes |

Abbreviations for amino acids:

| | |
|---|---|
| A | alanine |
| C | cysteine |
| D | aspartate |
| E | glutamate |
| F | phenylalanine |
| G | glycine |
| H | histidine |
| I | isoleucine |
| K | lysine |
| L | leucine |
| M | methionine |
| N | asparagine |
| P | proline |
| Q | glutamine |
| R | arginine |
| S | serine |
| T | threonine |
| V | valine |
| W | tryptophan |
| Y | tyrosine |

The figures show:

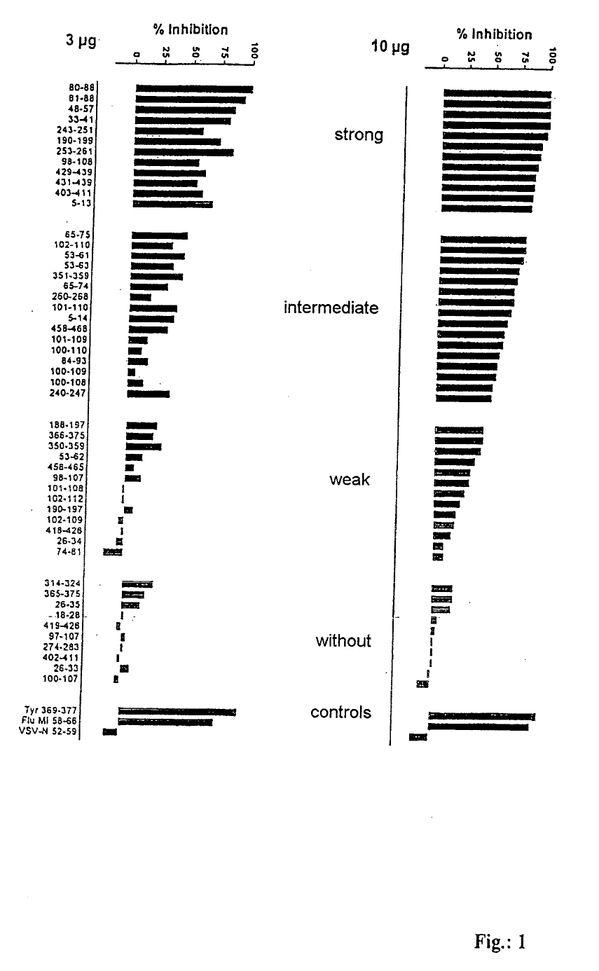

FIG. 1: Binding of selected synthetic hdm2 peptides.

The relative A2.1-binding affinity (indicated as % inhibition) was determined by the ability of the respective peptide to inhibit the A2.1 binding of the peptide p53 264-272. This was measured by means of the inhibition of the p53-specific CTL lysis of p53 264-272-loaded EA2 target cells by hdm2 peptides of differing concentration. The inhibition values for the peptides Flu M1 58-66 and VSV-N 52-59 were averaged from 7 independent experiments.

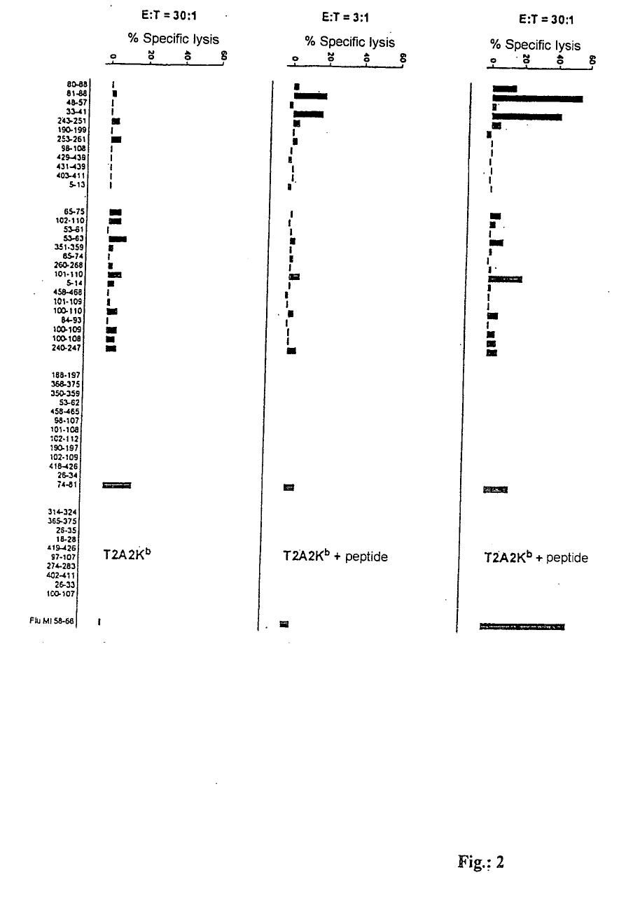

FIG. 2: A2.1-restricted immunogenicity of synthetic hdm2 peptides in A2K^{b}- or CD8×A2K^{b}-transgenic mice. The immunogenicity was checked by means of the lytic activity of the CTL induced in these mice by peptide immunization in a 4-hour cytotoxicity test. As target cells, T2A2K^{b} cells loaded with 2 μg of peptide or unloaded were employed. Representative specific lyses of individual CTL cultures from on average 4 immunized mice are shown.

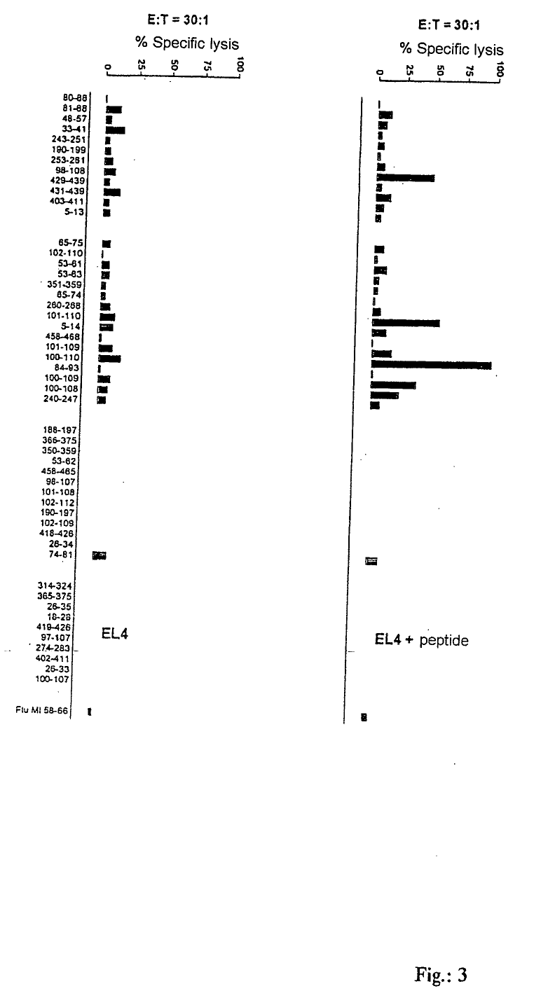

FIG. 3: H-2^{b}-restricted immunogenicity of synthetic hdm2 peptides in A2K^{b}- or CD8×A2K^{b}-transgenic mice. The immunogenicity was checked by means of the lytic activity of the CTL induced in these mice by peptide immunization in a 4-hour cytotoxicity test. As target cells, EL4 cells loaded with 2 μg of peptide or unloaded were employed. The data represent the specific lyses of the CTL cultures selected in FIG. 2.

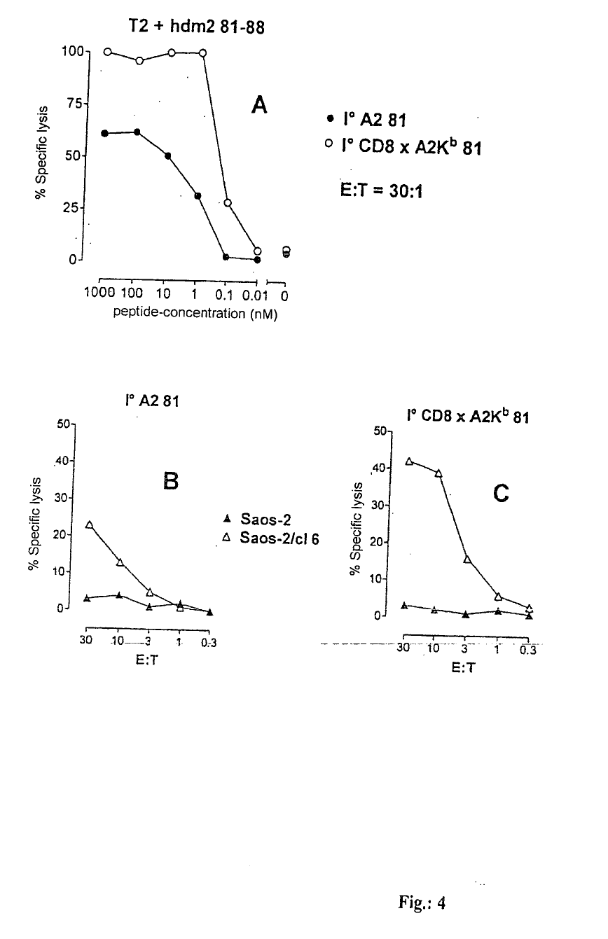

FIG. 4: The immunogenicity of the synthetic peptide hdm2 81-88 in A2.1- and CD8×A2K^{b}-transgenic mice. The lytic activity of the I° CTL A2 81 (●) and CD8×A2K^{b}81 (∘) induced in these mice by immunization with hdm2 81-88 was determined in a 6-hour cytotoxicity test. Target cells were: T2 cells (A) incubated at the peptide concentrations indicated, Saos-2 cells (▴) and hdm2-transfected Saos-2/cl 6 (Δ) (B, C).

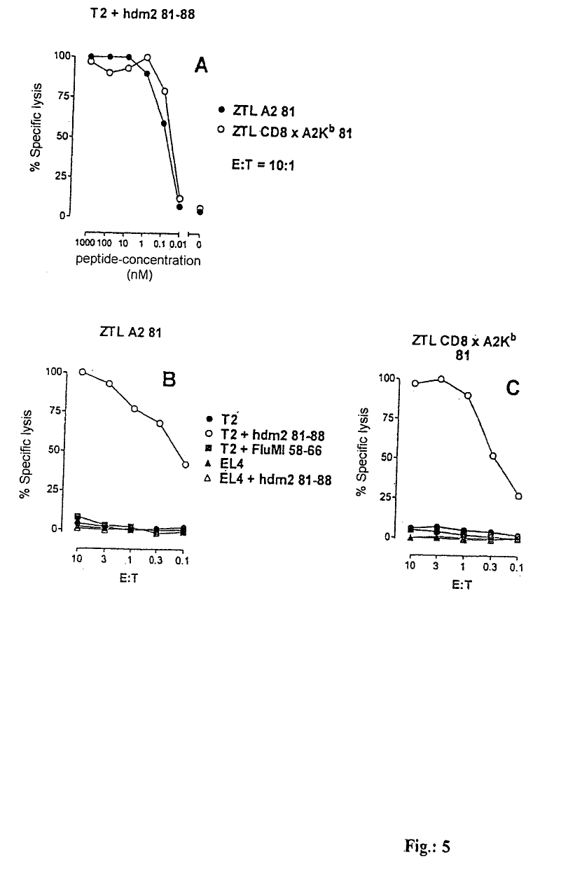

FIG. 5: hdm2 81-88-specific CTL lines: efficiency of the peptide recognition, peptide specificity and A2 restriction. The hdm2-reactive CTL lines A2 81 (●) and CD8×A2K^{b} 81 (∘) from A2.1- and CD8×A2 K^{b}-transgenic mice were established by repeated in vitro stimulation with hdm2 81-88-peptide and tested in a 4-hour cytotoxicity test. Target cells were: T2 cells incubated at the peptide concentrations indicated (A), hdm2 81-88-loaded (∘), Flu M1 58-66-loaded (▪) and unloaded (●) T2 target cells and hdm2 81-88-loaded (Δ) and unloaded (▴) EL4 cells (B, C).

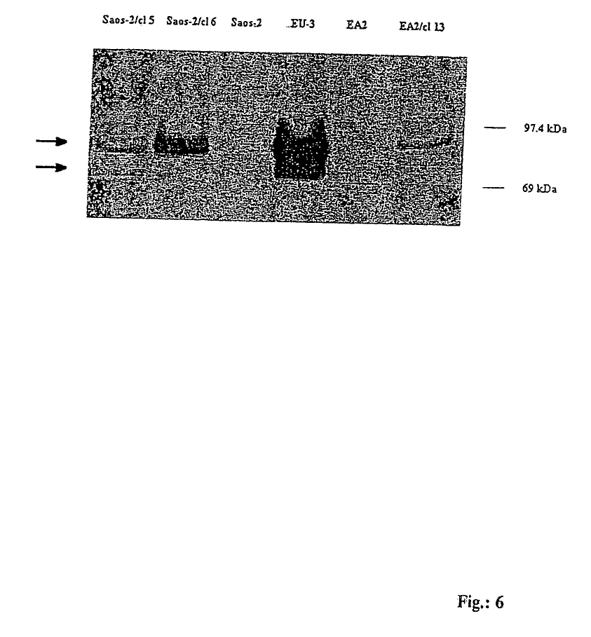

FIG. 6: hdm2-protein expression of hdm2 transfectants. The hdm2 transfectants Saos-2/cl 5 and 6 and EA2/cl 13 were generated by transfection of Saos-2 and EL4 cells. Nuclear extracts of these cells were separated electrophoretically, transferred to a membrane, incubated with an anti-hdm2 antibody and visualized photochemically. EU-3 functioned as a positive control. The arrows mark the 90 kDa full-length hdm2 protein and a 75 kDa hdm2 “splice” variant.

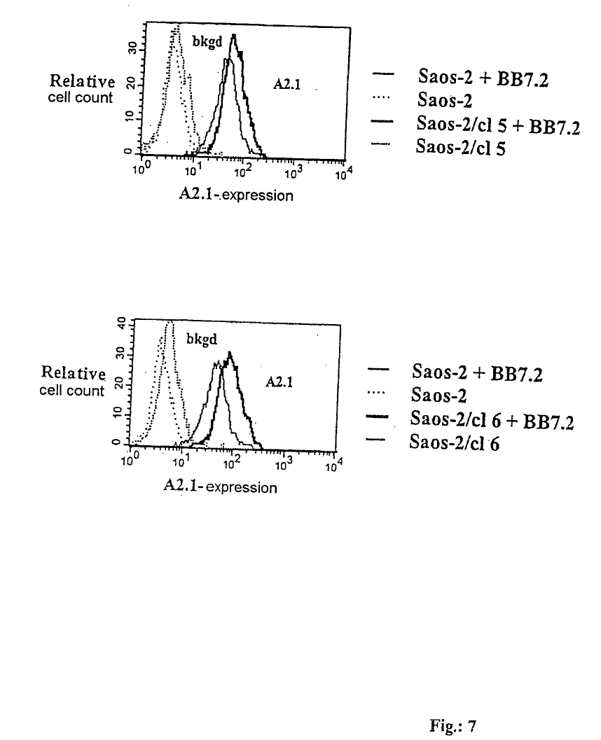

FIG. 7: A2.1 expression of Saos-2 hdm2 transfectants, Saos-2 cells and hdm2-transfected Saos-2/cl 5 and Saos-2/cl 6 cells were analyzed in the FACS with respect to their A2.1 expression after antibody labeling. The fluorescence intensities of the cells stained with the anti-A2.1 monoclonal antibody BB7.2 (A2.1) or serum (bkgd) and an FITC-conjugated secondary antibody are shown. The fluorescence intensity is indicated as A2.1 expression.

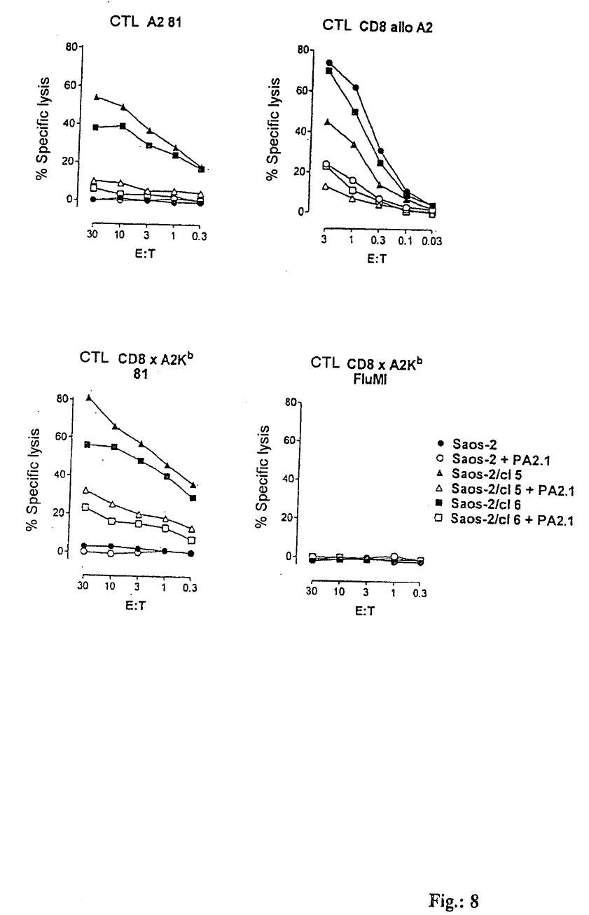

FIG. 8: CTL recognition von Saos-2 hdm2 transfectants. The A2.1-restricted and hdm2 81-88-specific CTL A2 and CD8×A2 K^{b} 81 and the allo-A2.1-reactive CTL CD8 allo A2 and the Flu M1 58-66-specific CTL CD8×A2 K^{b} Flu M1 were tested as effector cells under the E:T ratios indicated in a 6-hour cytotoxicity test against the following target cells: Saos-2 (●), hdm2-transfected Saos-2/cl 5 (▴) and Saos-2/cl 6 (▪); all target cells were treated with the anti-A2.1 monoclonal antibody PA2.1 as shown (∘, Δ, □).

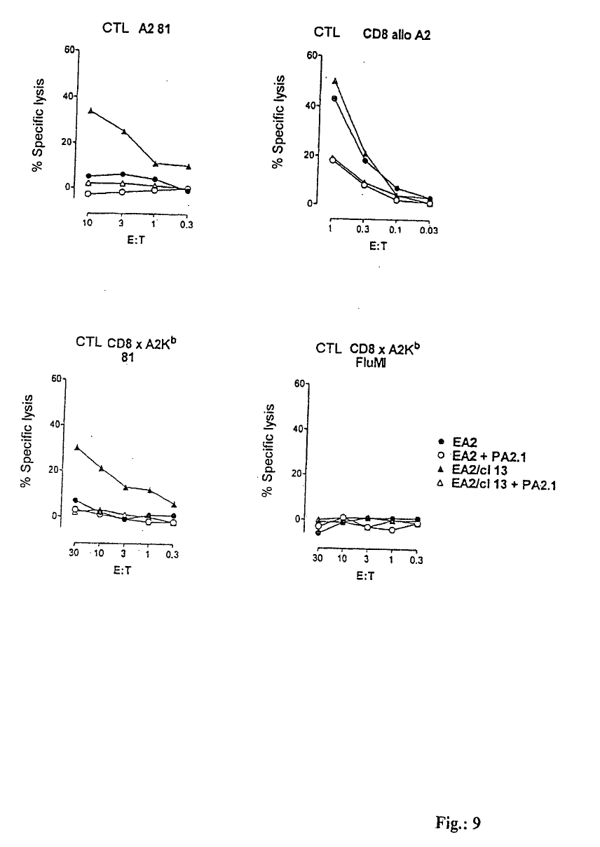

FIG. 9: CTL recognition of EA2 hdm2 transfectants. CTL A2 81, CTL CD8×A2K^{b} 81, CTL CD8 allo A2 and CTL CD8×A2 K^{b} Flu M1 were tested as effector cells under the E:T ratios indicated in a 6-hour cytotoxicity test against the following target cells: A2.1-transfected EL4 cells (EA2) (●) and EA2 cells, which were additionally cotransfected with the hdm2 gene (EA2/cl 13) (▴); the target cells were treated with the anti-A2.1 monoclonal antibody PA2.1 as shown (∘, Δ).

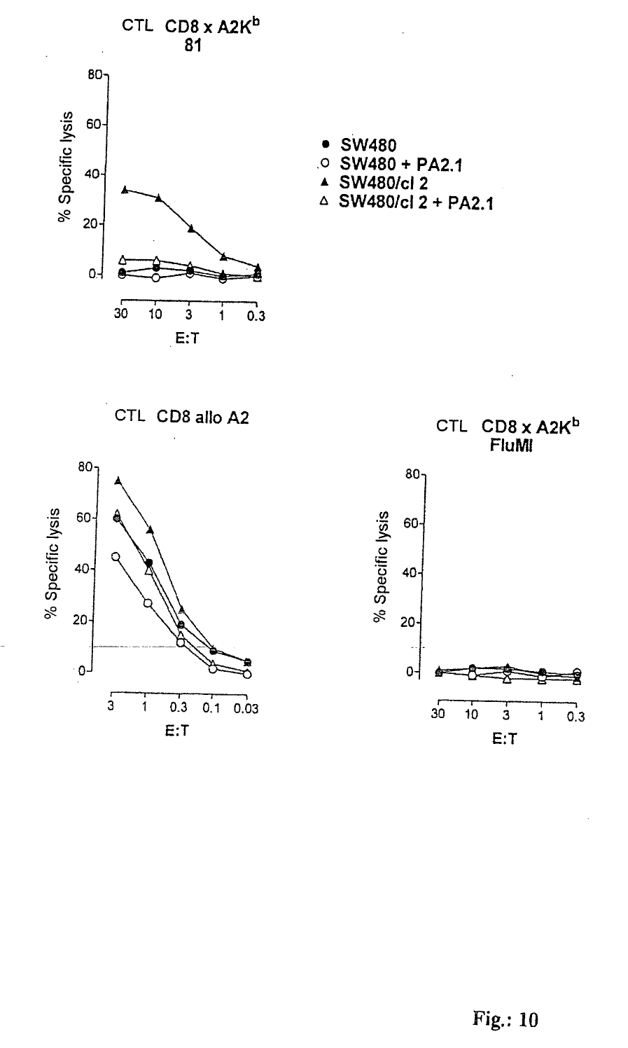

FIG. 10: CTL recognition of SW480 hdm2 transfectants. CTL CD8×A2 K^{b} 81, CTL CD8 allo A2 and CTL CD8×A2 K^{b} Flu M1 were tested as effector cells under the E:T ratios indicated in a 6-hour cytotoxicity test against the following target cells: SW480 cells (SW480) (●) and hdm2-transfected SW480 cells (SW480/cl 2) (▴); the target cells were treated with the anti-A2.1 monoclonal antibody PA2.1 as shown (∘, Δ).

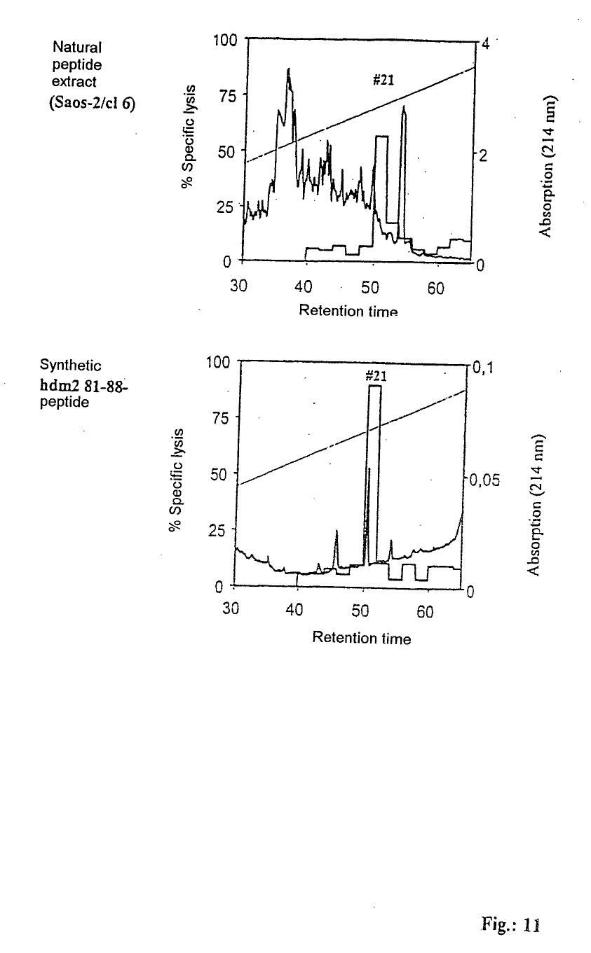

FIG. 11: The peptide hdm2 81-88 is the natural A2.1-presented epitope for hdm2-reactive CTL. Natural peptide extracts from MHC class I molecules of Saos-2/cl 6 and the synthetic hdm2 81-88 peptide were HPLC-fractionated and the individual HPLC fractions were incubated under serum-free conditions for 45 min with ⁵¹Cr-labeled T2 target cells. The loaded T2 cells were subjected to a 6-hour cytotoxicity test with CTL CD8×A2 K^{b} 81 at an E:T ratio of 20:1. The HPLC profile (absorption at 214 nm) and the specific lysis (bar) of the T2 target cells loaded with the individual HPLC fractions are shown as a function of the retention time.

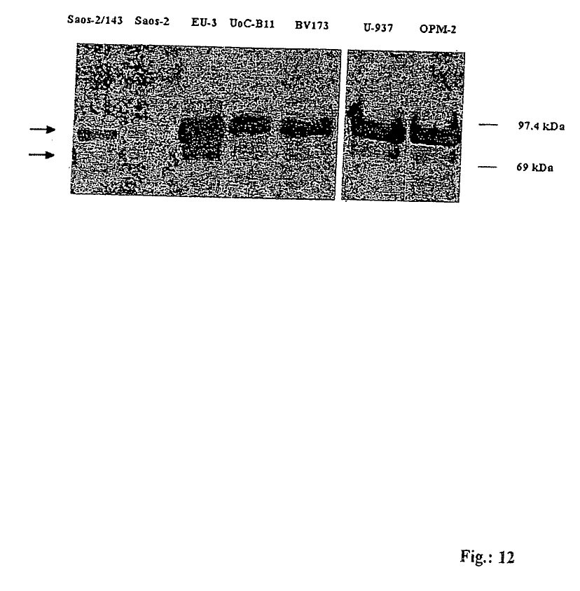

FIG. 12: hdm2 protein expression of human A2-positive tumor cell lines. Nuclear extracts of EU-3, UoC-B11 and BV173 (pre-B-ALL) and U-937 (histiocytic lymphoma) and OPM-2 (plasmocytoma) were prepared, separated by gel electrophoresis, transferred to a membrane, incubated with an anti-hdm2 antibody and visualized photochemically. The arrows mark the 90 kDa full-length hdm2 protein and a 75 kDa hdm2 “splice” variant.

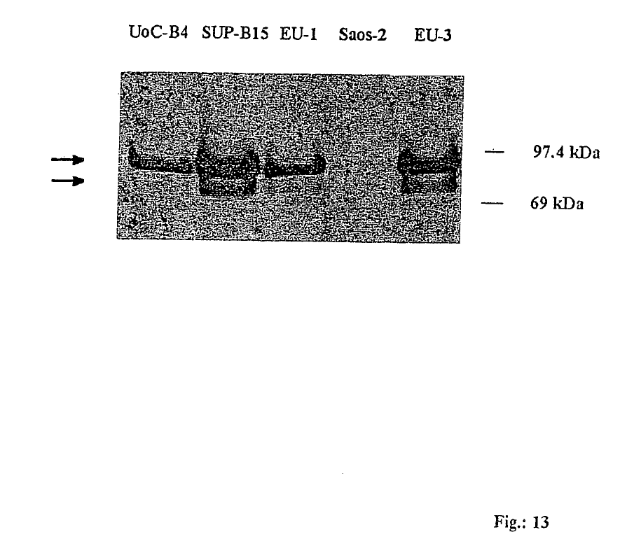

FIG. 13: hdm2 protein expression of human A2-negative tumor cell lines. Nuclear extracts of the B-ALL lines UoC-B4, SUP-B15 and EU-1 were prepared, separated by gel electrophoresis, transferred to a membrane, incubated with an anti-hdm2 antibody and visualized photochemically. EU-3 functioned as a positive control, Saos-2 as a negative control.

The arrows mark the 90 kDa full-length hdm2 protein and a 75 kDa hdm2 “splice” variant.

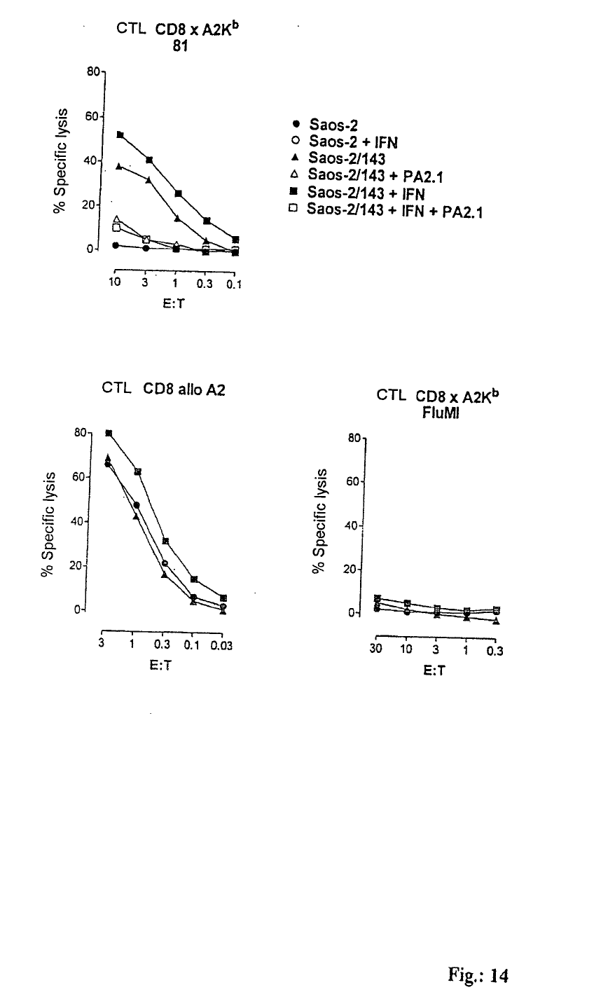

FIG. 14: CTL recognition of p53/143-transfected Saos-2 cells. The A2.1-restricted and hdm2 81-88-specific CTL CD8×A2 K^{b} 81 and the allo-A2.1-reactive CTL CD8 allo A2 and the Flu M1 58-66-specific CTL CD8×A2 K^{b} Flu M1 were tested as effector cells under the E:T ratios indicated in a 6-hour cytotoxicity test against the following target cells: Saos-2 with (∘) and without (●) IFN-γ treatment (20 ng/ml for 20 h) and p53/143-transfected Saos-2 cells (Saos-2/143) with (▪) and without (▴) IFN-γ treatment. Saos-2/143 cells were treated with the anti-A2.1 monoclonal antibody PA2.1 as shown (Δ, □).

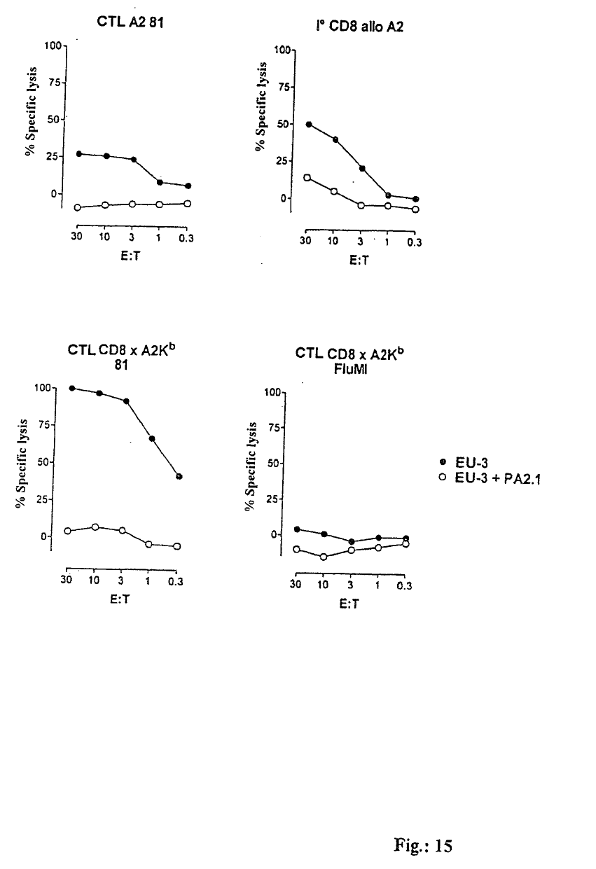

FIG. 15: CTL recognition of the hdm2-overexpressing A2-positive tumor cell line EU-3. CTL A2 81, CTL CD8×A2 K^{b} 81, I° CTL CD8 allo A2 and CTL CD8×A2 K^{b} Flu M1 were tested as effector cells under the E:T ratios indicated in a 6-hour cytotoxicity test against the pre-B ALL cell line EU-3 with (∘) and without (●) PA2.1.

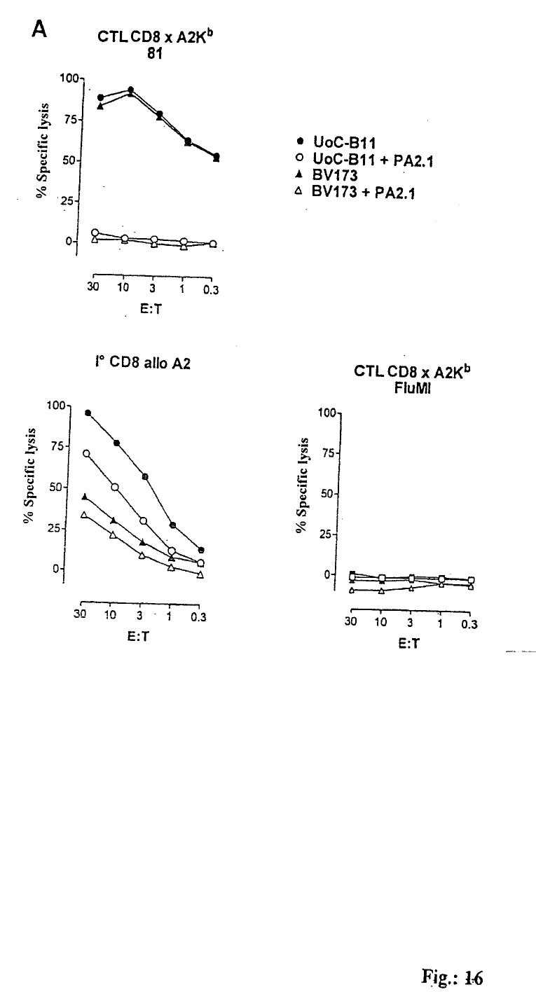

FIG. 16: CTL recognition of hdm2-overexpressing A2-positive leukemia cell lines. CTL CD8×A2 K^{b} 81, I° CTL CD8 allo A2 and CTL CD8×A2 K^{b} Flu M1 were tested as effector cells under the E:T ratios indicated in a 6-hour cytotoxicity test against the target cells UoC-B11 (●) and BV173 (▴) (pre-B-ALL). All target cells were treated with the anti-A2.1 monoclonal antibody PA2.1 as shown (∘, Δ).

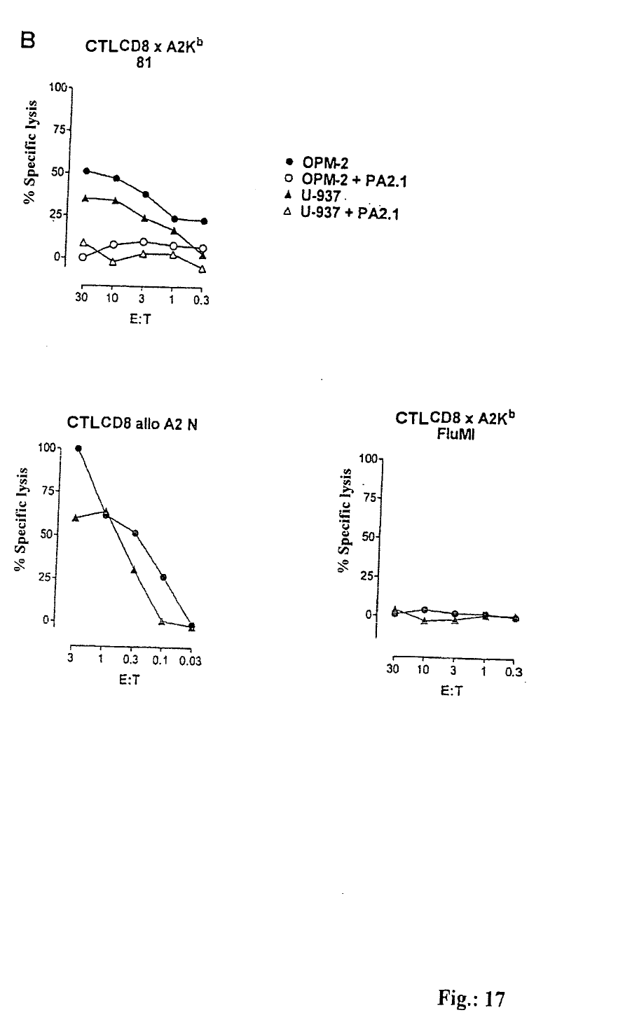

FIG. 17: CTL recognition of hdm2-overexpressing A2-positive lymphoma and plasmocytoma cell lines. CTL CD8×A2 K^{b} 81, CTL CD8 allo A2 N and CTL CD8×A2 K^{b} Flu M1 were tested as effector cells under the E:T ratios indicated in a 6-hour cytotoxicity test against the target cells OPM-2 (●) (plasmocytoma) and U-937 (▴) (histiocytic lymphoma). All target cells were treated with the anti-A2.1 monoclonal antibody PA2.1 as shown (∘, Δ).

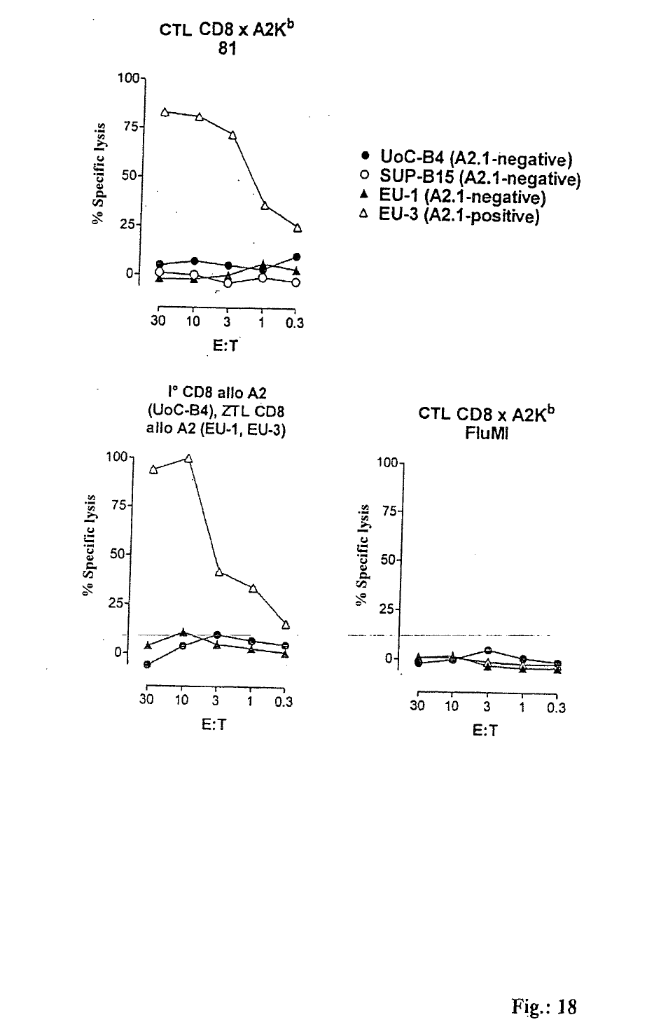

FIG. 18: A2-negative hdm2-overexpressing leukemia cell lines are not recognized. CTL CD8×A2 K^{b} 81, allo-A2.1-reactive CTL and CTL CD8×A2 K^{b} Flu M1 were tested as effector cells under the E:T ratios indicated in a 6-hour cytotoxicity test against the pre-B-ALL cell lines UoC-B4 (●), EU-1 (▴) and SUP-B15 (∘). The A2-positive pre-B-ALL line EU-3 (Δ) functioned as a positive control.

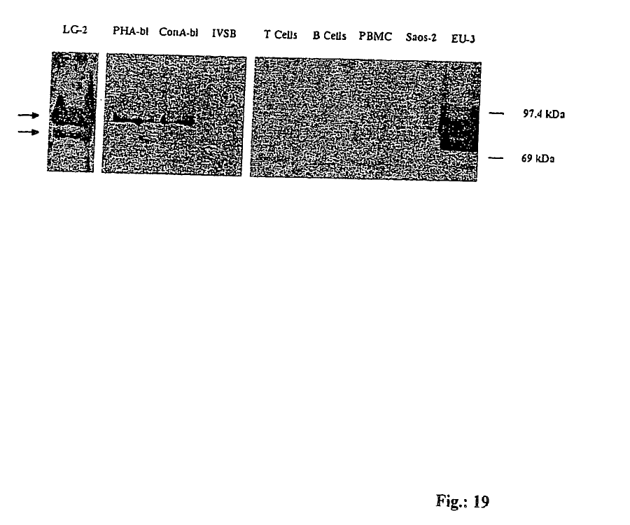

FIG. 19: hdm2 protein expression of lymphohemopoietic cells. The following cells were investigated: the EBV-LCL LG-2, PHA and Con A blasts, the tyrosinase-specific CTL clone IVSB, resting T and B cells, resting PBMC. Nuclear extracts were prepared, separated by gel electrophoresis, transferred to a membrane, labeled with an anti-hdm2 antibody and visualized photochemically. EU-3 functioned as a positive control, Saos-2 as a negative control. The arrows mark the 90 kDa full-length hdm2 protein and a 75 kDa hdm2 “splice” variant.

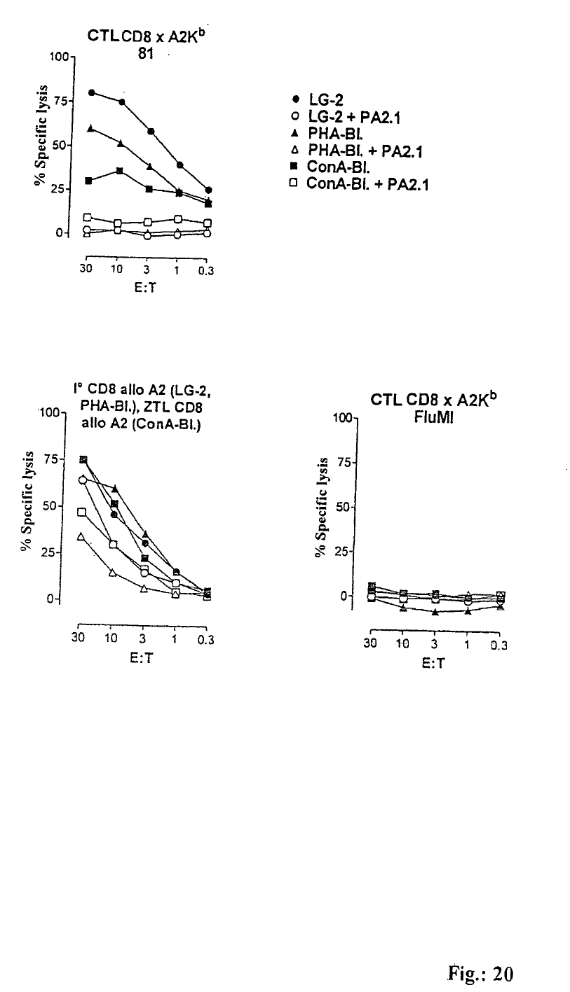

FIG. 20: CTL recognition of transformed lymphohemopoietic cells. CTL CD8×A2 K^{b} 81, allo-A2.1-reactive CTL and CTL CD8×A2 K^{b} Flu M1 were tested as effector cells under the E:T ratios indicated in a 6-hour cytotoxicity test against the following target cells: EBV-LCL LG-2 (●), PHA blasts (▴) and Con A blasts (▪). All target cells were treated with the anti-A2.1 monoclonal antibody PA2.1 as shown (∘, Δ, □).

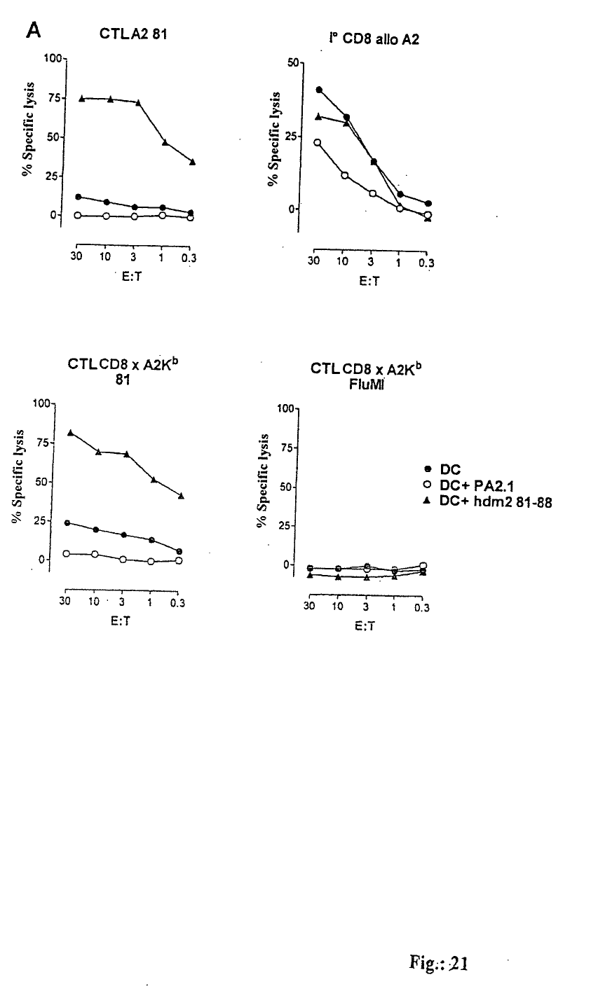

FIG. 21: Absence of substantial recognition of activated mature dendritic cells (DC). CTL A2 81, CTL CD8×A2 K^{b} 81, allo-A2.1-reactive CTL and CTL CD8×A2 K^{b} Flu M1 were tested as effector cells under the E:T ratios indicated in a 6-hour cytotoxicity test against activated mature DC (●) and the same cells loaded with hdm2 81-88 peptide (10 μM) (▴). The target cells were treated with the anti-A2.1 monoclonal antibody PA2.1 as shown (∘).

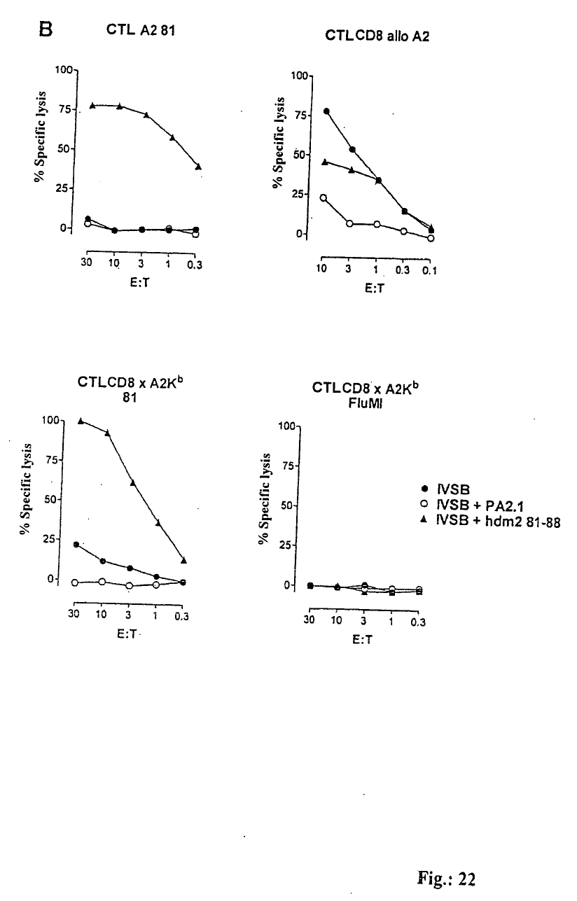

FIG. 22: Absence of substantial recognition of antigen-activated T cells. CTL A2 81, CTL CD8×A2 K^{b} 81, allo-A2.1-reactive CTL and CTL CD8×A2 K^{b} Flu M1 were tested as effector cells under the E:T ratios indicated in a 6-hour cytotoxicity test against tyrosinase-specific CTL clone IVSB (●) and the same cells loaded with hdm2 81-88 peptide (10 μM) (▴). The target cells were treated with the anti-A2.1 monoclonal antibody PA2.1 as shown (∘).

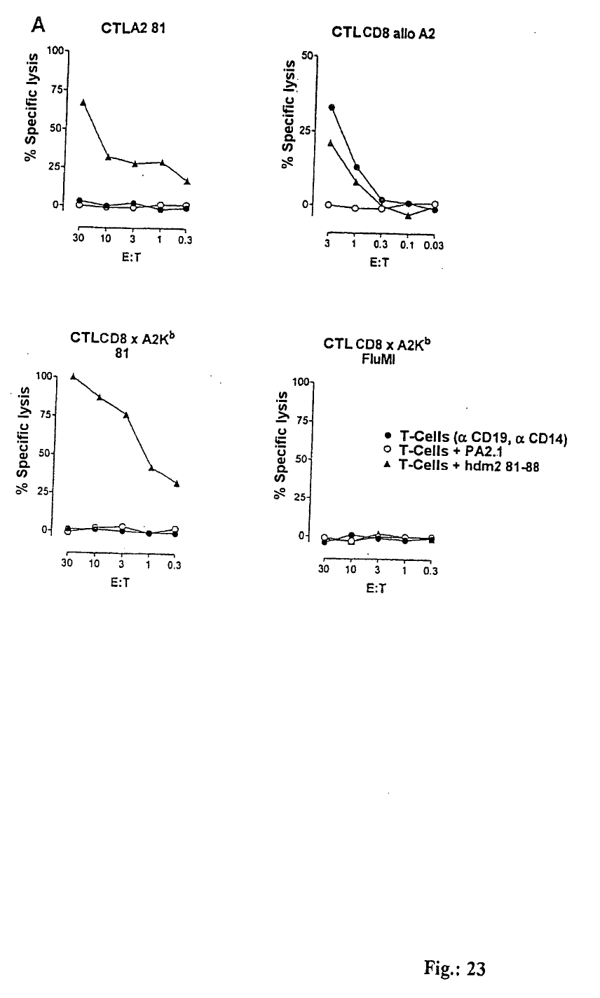

FIG. 23: Resting lymphohemopoietic cells are not recognized. CTL A2 81, CTL CD8×A2 K^{b} 81, CTL CD8 allo A2 and CTL CD8×A2 K^{b} Flu M1 were tested as effector cells under the E:T ratios indicated in a 6-hour cytotoxicity test against resting T cells (●) and the same cells loaded with hdm2 81-88 peptide (10 μM) (▴). The target cells were treated with the anti-A2.1 monoclonal antibody PA2.1 as shown (∘).

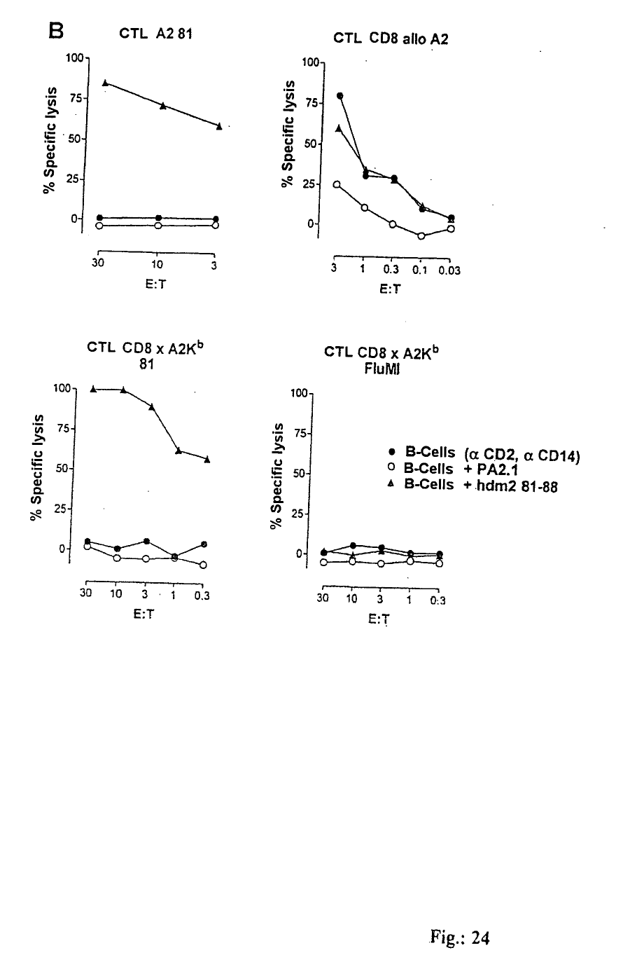

FIG. 24: Resting lymphohemopoietic cells are not recognized. CTL A2 81, CTL CD8×A2 K^{b} 81, CTL CD8 allo A2 and CTL CD8×A2K^{b} Flu M1 were tested as effector cells under the E:T ratios indicated in a 6-hour cytotoxicity test against resting B cells (●) and the same cells loaded with hdm2 81-88 peptide (10 μM) (▴). The target cells were treated with the anti-A2.1 monoclonal antibody PA2.1 as shown (∘).

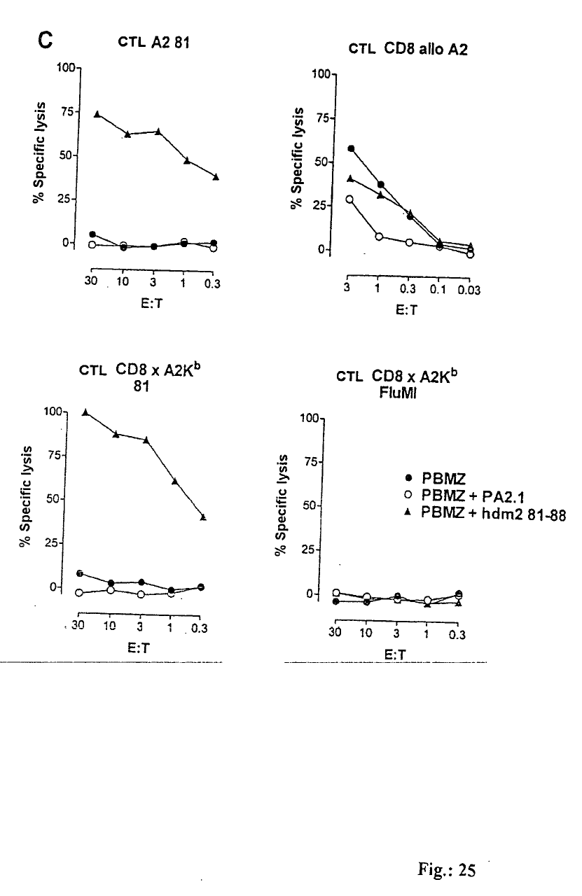

FIG. 25: Resting lymphohemopoietic cells are not recognized. CTL A2 81, CTL CD8×A2 K^{b} 81, CTL CD8 allo A2 and CTL. CD8×A2 K^{b} Flu M1 were tested as effector cells under the E:T ratios indicated in a 6-hour cytotoxicity test against PBMC (●) and the same cells loaded with hdm2 81-88 peptide (10 μM) (▴). The target cells were treated with the anti-A2.1 monoclonal antibody PA2.1 as shown (∘).

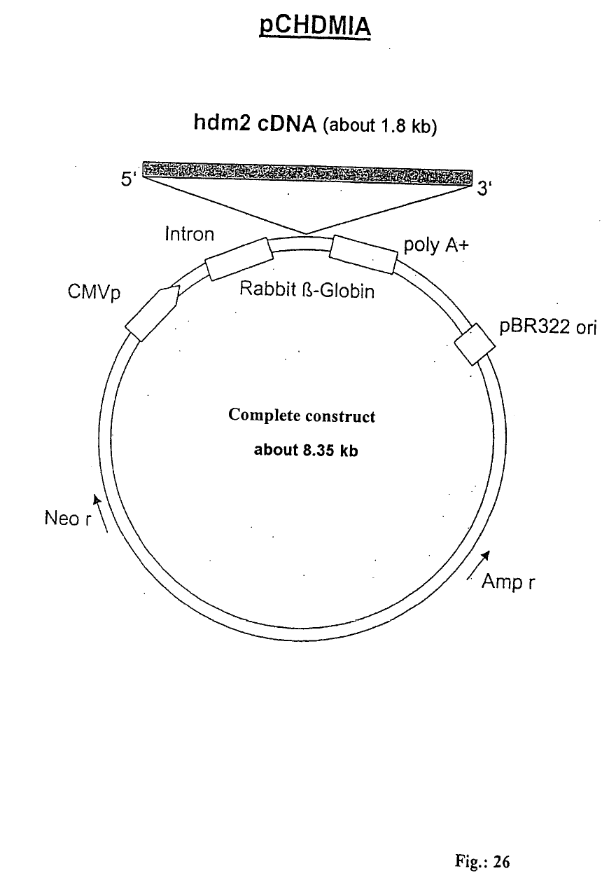

FIG. 26: Plasmid pCHDMIA coding for the hdm 2 protein 2.

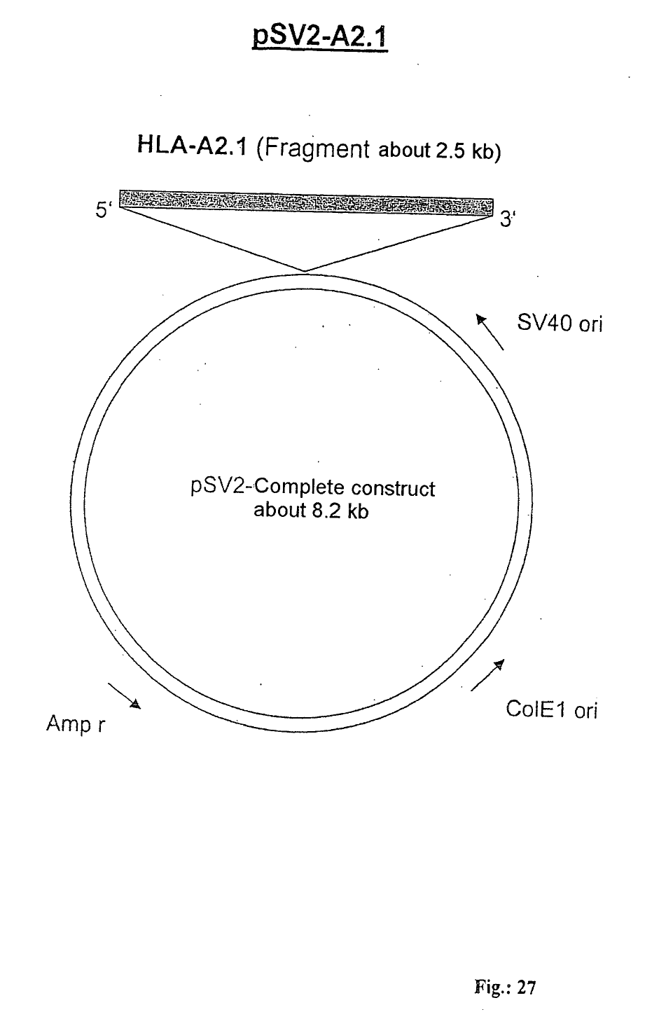

FIG. 27: Plasmid pSV2-A2.1 coding for A2.1

### A) MATERIALS MENTIONED IN THE EXAMPLES

(1) Mice

Transgenic mice which express the human MHC class I transgene HLA-A2.1 (A2.1) were crossed into the C57BL/6 background using technically customary methods (Irwin et al., 1989). The following strains were used for this:

1) A2.1/K^{b} (A2 K^{b})-transgenic mice—they are homozygous for a chimeric MHC class I transgene which is composed of the human α₁, and α₂ domains of A2.1 and of the α₃ domain of H-2 K^{b} of the mouse, and also for the H-2^{b} gene.

2) huCD8α/β (CD8)-transgenic mice—they are homozygous for the α- and β-chain of the human CD8 coreceptor.

3) [huCD8α/β×A2.1/K^{b}]_{F1} (CD8×A2K^{b})-transgenic mice—they heterozygously express the chimeric A2 K^{b} molecule and additionally the α- and β-chain of the human CD8. They are moreover homozygous for H-2^{b}.

4) A2.1-transgenic mice (([A2.1×C57BL/6]×C57BL/6)_{F1}-transgenic)—they express the α₁, α₂ and α₃ domains of the human A2.1 molecule heterozygously and are homozygous for H-2^{b}.

5) C57BL/6 mice—they possess the H-2^{b} phenotype.

(2) Synthetic Peptides

Synthetic peptides were obtained from the Scripps Research Institute and from SNPE (Neosystem laboratoire, Strasbourg, France). The purity of the peptides synthesized by the Scripps Research Institute using the automatic peptide synthesis apparatus 430A (Applied Biosystems, Foster City, Calif.) was at least 70%, the purity of the peptides synthesized by SNPE at least 75%. The purity and correct amino acid composition of all peptides was checked by HPLC analysis and by mass spectrometry. Lyophilized and demineralized peptides from the Scripps Research Institute were dissolved to 10 mg/ml in DMSO, H₂O, mixtures of DMSO and H₂O, or in 0.1% strength NaOH according to quantitative control as a function of the peptide sequence. Nondemineralized peptides of SNPE were basically dissolved to 10 mg/ml in DMSO. Storage took place in aliquots at −20 to −80° C. Additionally to the peptides shown in Tab. 1, a peptide which represents the residues 128-140 of the hepatitis B virus core protein was synthesized (TPPAYRPPNAPIL).

(3) Antibodies

For the blockade of A2.1, the monoclonal antibody produced by the hybridoma cell line PA2.1 (ATCC HB-117) was used.

For the HLA typing of tumor cell lines and of A2-transgenic mice, the monoclonal antibody produced from the mouse hybridoma line BB7.2 (ATCC HB-82) was employed.

For the analysis of the hdm2 expression of cells, the commercially obtainable anti-hdm2 monoclonal antibody IF2 (mouse IgG_{2b}) (Oncogene Research Products, Cambridge, Mass.) was used.

For the detection of monoclonal antibodies of the mouse in flow cytometry, an FITC-conjugated polyclonal secondary antibody (goat anti-mouse IgG F(ab)₂ fragment; 1:30 dilution; Jackson [Dianova], Hamburg) was employed. The detection of the monoclonal antibody IF2 was carried out using a peroxidase (horseradish peroxidase)-conjugated secondary antibody (goat anti-mouse IgG; Pierce, Ill.).

(4) Cells, Cell Lines and Transfectants

All cells and cell lines were cultured in RPMI 1640 (Biowhittaker, Verviers, Belgium) in the presence of 10% of heat-inactivated (30 min, 56° C.) FCS (PAA Laboratories, Linz, Austria), 1% of 0.2 M L-glutamine (Biowhittaker) and 50 μg/ml of gentamycin (Gibco BRL, Eggenstein). For the propagation of cells and CTL lines from the mouse, β-mercaptoethanol was additionally added to the medium in a final concentration of 5×10⁻⁵ M. For the cultivation of neomycin-transfected cells, geneticin (G-418) (Gibco BRL) was added to the medium in an effective concentration of 280-560 μg/ml. All cells were cultured at 37° C. and under 5% CO₂ in a water vapor-saturated atmosphere in cell culture bottles or 24-well plates (CTL) (Corning Costar, Bodenheim).

(4.1) Cells: For the obtainment of mononuclear cells of the peripheral blood (PBMC), the blood of a healthy A2-positive donor was diluted with PBS (Biowhittaker, Walkersville, Mass.) in the ratio 1:3 and underlaid with the same volume of Ficoll (Seromed Biochrom, Berlin). After centrifugation (1500 rpm, 5° C., 7 min), the PBMC were isolated from the interphase and washed.

Con A- and PHA-activated lymphoblasts were generated using technically customary processes (cf. Theobald et al., 1995) by 3-days' stimulation of A2-positive PBMC with Con A (10 μg/ml) and PHA (1.5% w/v) (Gibco BRL, Eggenstein).

The obtainment of resting T and B cells was carried out by negative selection of A2-positive PBMC using antibody-coated beads (Dynal, Hamburg). For the isolation of T cells, the PBMC were incubated with anti-CD19 and anti-CD14 beads according to the instructions of the manufacturer, for the isolation of B cells with anti-CD2 and anti-CD14 beads.

Dendritic cells (DC) were generated from PBMC of an A2-positive donor using technically customary methods. After incubation of the PBMC for 45 min at 37° C. in a petri dish, nonadherent cells were rinsed off and the adherent PBMC were taken up in X-Vivo 15 (Biowhittaker, Verviers, Belgium), which was supplemented with 1.5% of autologous heat-inactivated plasma, 1000 U/ml of IL-4 (PBH Strathmann Biotech, Hanover) and 800 U/ml of GM-CSF (“Leucomax”, Sandoz, Nuremberg) (Jonuleit et al., 1997). On day 3 and 5, a partial change of medium was carried out with addition of 1000 U/ml of IL-4 and 1600 U/ml of GM-CSF, but without autologous plasma. The adherent PBMC differentiated to give nonadherent dendriphages. On day 7, these immature DC were inoculated in X-Vivo 15 with 1.5% of autologous plasma and treated with 500 U/ml of IL-4, 800 U/ml of GM-CSF, 10 ng/ml of TNF-α (Genzyme, Cambridge, Mass.), 10 ng/ml of IL-1β (PBH Strathmann Biotech), 1000 U/ml of IL-6 (PBH Strathmann Biotech) and 1 μg/ml of PG-E₂ (“Minprostin E2”; Pharmacia Biotech, Freiburg) (Jonuleit et al., 1997). The mature DC expressed HLA-DR, CD58, CD80, CD83 and CD86 on day 9 and 10.

An A2.1-positive CTL clone “IVSB” having specificity for the tyrosinase peptide 369-377 was produced and made available using technically customary methods.

All cells mentioned served as target cells in the cytotoxicity test (“CTL recognition”).

(4.2) Cell lines and transfectants: The cell lines and transfectants listed below, prepared according to (4.1) or known in the prior art and obtainable at any time, were employed for the investigations described here:

the human A2.1-positive T2 cell line is a B/T cell hybridoma of the fusion partners 721.147 and CEM (Salter and Cresswell, 1986),

T2 cells which were transfected with the A2 K^{b} gene according to Theobald et al., 1995 (T2A2 K^{b}),

the thymoma cell line EL4 from the C57BL/6 mouse (Theobald et al., 1995),

EL4 cells which were transfected with A2.1 (EA2) (Theobald et al., 1995),

the human T-cell leukemia line Jurkat (Theobald et al., 1995),

Jurkat cells which were transfected with A2.1 (JA2) (Theobald et al., 1995),

the constitutively A2.1-positive and p53-defect-mutant osteosarcoma cell line Saos-2 (Dittmer et al., 1993)

Saos-2 cells which were transfected with human p53 gene, which has a mutation on residue 143 (V→A) (Dittmer et al., 1993);

the human hdm2-overexpressing leukemia line EU-3 (Pre-B-ALL, A2-positive)(Zhou et al., 1995)

the human hdm2-overexpressing leukemia line UoC-B11 (Pre-B-ALL, A2-positive) (Zhou et al., 1995)

the human hdm2-overexpressing leukemia line EU-1 (Pre-B-ALL, A2-negative)(Zhou et al., 1995),

the human hdm2-overexpressing leukemia line UoC-B4 (Pre-B-ALL, A2-negative) (Zhou et al., 1995),

the human hdm2-overexpressing leukemia line SUP-B15 (Pre-B-ALL, A2-negative) (Zhou et al., 1995),

the A2-positive cell line Pre-B-ALL BV173 (DSM ACC 20; DSMZ, Braunschweig, Germany),

the A2-positive histiocytic lymphoma cell line U-937 (ATCC CRL-1593; Rockville, Mass., USA),

the A2-positive cell line plasmocytoma OPM-2 (DSM ACC 50, DSMZ, Braunschweig, Germany)

the EBV-transformed lymphoblastoid and A2-positive cell line LG-2

the human A2-positive colon carcinoma cell line SW480 (DKFZ, Heidelberg, FRG).

All cells mentioned served as target cells in the cytotoxicity test. The Saos-2 and Saos-2/143 target cells were pretreated for cytotoxicity tests with recombinant IFN-γ (R&D Systems, Minneapolis, Minn.) in a concentration of 20 ng/ml for 20 hours.

B) Methods Used in the Examples

(1) Transfection

(1.1) Molecular Biology Methods

In order stably to transfect mammalian cells with the hdm2 or A2.1 gene, the plasmid pCHDMIA according to FIG. 26 (cf. Wu et al., 1993) coding for hdm2 and the plasmid pSV2-A2.1 according to FIG. 27 (cf. Irwin et al., 1989) coding for A2.1 were employed. The pCHDMIA plasmid additionally codes for neomycin and ampicillin resistance, the pSV2-A2.1 plasmid additionally for ampicillin resistance. The hdm2 cDNA is under the control of the CMV promoter, the A2.1 cDNA under the control of the SV-40 promoter.

For the transformation of Escherichia coli with plasmid DNA, competent cells of the E. coli strain DH5α were prepared using processes familiar to the person skilled in the art. DNA was added to the competent bacterial cells and, after 15 minutes' incubation on ice, the cells were exposed to a heat shock for 2 min at 42° C. After addition of LB medium (10 g of tryptone, 5 g of yeast extract, 10 g of NaCl, H₂O to 1000 ml, pH 7.5), the batch was incubated at 37° C. for 20 min and finally plated out on LB agar plates (1.5% w/v Japan agar; Merck, Darmstadt) in the presence of 100 μg/ml of ampicillin (Boehringer Mannheim, Mannheim) and incubated at 37° C. Single colonies were picked, inoculated into LB medium with ampicillin and incubated at 37° C. with shaking (220 rpm) (preculture). The cells were then harvested and subjected to a plasmid preparation. The preparation was carried out using a “QIAprep Spin Miniprep Kit” according to the instructions of the manufacturer (Qiagen, Hilden). Plasmid-bearing transformants were identified by means of restriction analysis using suitable restriction endonucleases and subsequent agarose gel electrophoresis. The gel material used was 0.6-1.5% strength agarose (w/v), which was prepared in TBE buffer (50 nM tris borate, 2.5 mM Na₂-EDTA, pH 8.5). The positive transformants were then cultured overnight at 37° C. on a larger scale (main culture) in ampicillin-containing LB medium. After cell harvesting, the plasmids were prepared using a “QIAGEN Plasmid Maxi Kit” according to the manufacturer's instructions (Qiagen). The resulting DNA solution was checked photometrically for its concentration and purity by measurement of the absorption (OD) at a wavelength of 260 nm and 280 nm in quartz cuvettes. After fresh analytical restriction and agarose gel electrophoresis, the DNA was linearized for the electroporation, but not for the lipofection. The plasmid pCHDMIA was cleaved using the restriction endonuclease PvuI (MBI Fermentas, St. Leon Rot) with addition of BSA (0.2 mg/ml) and the pSV2-A2.1 plasmid was cleaved using EcoRI (MBI Fermentas). For the checking of the restriction, the samples were analyzed by gel electrophoresis. In order to eliminate the restriction endonucleases from the DNA solutions, an extraction was carried out. For this, the samples were treated with one volume of phenol/chloroform/isoamyl alcohol (24:24:1, v/v/v; Roth, Karlsruhe) and centrifuged after thorough mixing (14000 rpm, 4 min, room temperature). The DNA-containing aqueous upper phase was isolated and subjected to a fresh extraction. For the precipitation of the DNA, the DNA solution was treated with 1/10 volume of Na acetate (3 M) and, after mixing, with 2 volumes of ethanol (96%, v/v, −20° C.). Following a one-hour incubation at −20° C., the samples were centrifuged off for 20 min at 4° C. and briefly washed with approximately 2 volumes of ethanol (70%, v/v, −20° C.). After drying the DNA pellets in air, the DNA was dissolved in TE buffer (10 mM tris, 1 mM Na₂-EDTA, pH 8) and stored at −20° C.

(1.2) Transfection Methods

For the stable transfection of mammalian cells, DNA of high purity was employed, which had an OD quotient 260/280 nm of at least 1.8.

a) Lipofection: The adherent Saos-2 and SW480 cells were cultured in petri dishes (Greiner, Frickenhausen) and were confluent to 30-50% on the day of transfection (about 15 Mio cells/78 cm² dish). The procedure was carried out using a commercially obtainable lipofection kit (Gibco BRL, Eggenstein) modified according to the instructions of the manufacturer. In 12 ml snap-lid tubes made of polystyrene (Corning Costar, Bodenheim), 30 μg of DNA were mixed with 1.5 ml of Opti-Mem I (Gibco BRL) (batch A) or 60 μl of lipofectin (Gibco BRL) with 0.3 ml of Opti-Mem I (batch B) and incubated at room temperature for one hour. The batches A and B were mixed (A/B) and incubated for a further 10-15 min. RPMI 1640 (1% glutamine) (Biowhittaker, Verviers, Belgium) was then added to the batch A/B in a final volume of altogether 2-6 ml. This DNA- and lipofectin-containing solution was distributed over the cells washed intermediately with RPMI 1640 (1% glutamine) after mixing. After at least 5-hours' incubation at 37° C. and 5% CO₂ with water vapor saturation, the DNA-containing medium was taken off and the cells were overlaid with 10 ml of cell culture medium (see 2.4). Following a further incubation for about 24 hours, the transfected cells were selected 1:2 in selection medium (cell culture medium containing 0.56 mg/ml of G-418 [Gibco BRL]). A change of the selection medium was carried out twice per week. After 3-4 weeks, after repeated washing of the petri dishes with PBS (Biowhittaker, Walkersville, Mass.), neomycin-resistant clones were isolated and transferred to a 48-well plate. The transfectants were finally transferred to cell culture bottles and tested for their hdm2 and A2.1 expression.

b) Elektroporation: For the cotransfection of the suspension cell line EL4 with pCHDMIA and pSV2-A2.1 plasmids, 10 Mio EL4 cells were washed, resuspended in 0.5 ml of RPMI 1640 (Biowhittaker, Verviers, Belgium) and 1% of FCS (PAA Laboratories, Linz, Austria) and pipetted into 4 mm cuvettes (BioRad Laboratories, Munich). 20 μg of linearized DNA of the pSV2-A2.1 plasmid and 4.5 μg of the linearized pCHDMIA plasmid were mixed and then added to the cells. The cells were electroporated at 1200 μFarad and 300 volts for 2 ms in a “Gene Pulser” (Fischer, Heidelberg). The cells were then serially diluted with cell culture medium (see 2.4) in 96-well plates and cultured for 24 hours at 37° C. and 5% CO₂ with water vapor saturation. The addition of G-418 (Gibco BRL, Eggenstein) was carried out in an effective final concentration of 560 μg/ml. A change of the selection medium was carried out weekly. After approximately 2-3 weeks, the neomycin-resistant transfectant clones were transferred, firstly to 24-well plates, later to cell culture bottles, until they were finally checked for the expression of hdm2 and A2.1.

(2) Flow Cytometry

The A2.1 expression of cells, cell lines and trans-fectants was measured in a fluorescence-activated cell sorter (FACS) (Becton Dickinson, San Jose, Calif.). In each case, 0.5 Mio cells were centrifuged off and labeled with the anti-A2.1 monoclonal antibody BB7.2 (or RPMI 1640, 10% FCS, see 2.4) in a volume of 50 μl (Lustgarten et al., 1997). After one hour's incubation on ice, the batches were washed twice with PBS (Biowhittaker, Walkersville, Mass.) and the cells then counterstained with an FITC-conjugated secondary antibody (goat anti-mouse IgG F_{ab} fragment; 50 μl of a 1:30 dilution in PBS). After incubation on ice for 25 min, the samples were washed twice with PBS and finally fixed in PBS and 1% formalin. The fluorescence activity of the cell populations selected in the forward-scattered light was determined in the FACS.

(3) Western Blot

a) Nuclear protein extraction: All working steps were carried out at 4° C. The cells were washed twice with PBS (Biowhittaker, Walkersville, Mass.) (1500 rpm, 5° C., 7 min) and then resuspended in buffer A (10 mM HEPES, pH 7.9, 1.5 mM MgCl₂, 10 mM KCl) in the presence of protease inhibitors (see below). For the lysis of the cell membranes, 2 μl of buffer A/Mio suspension cells or 4 μl buffer A/Mio adherent cells were employed. The solution was centrifuged on ice after incubation for 10 min (14000 rpm, 4° C., 10 s). After taking up again, incubation and centrifugation, the cell nuclei were resuspended in buffer C (20 mM HEPES, pH 7.9, 1.5 mM MgCl₂, 0.42 M NaCl, 0.2 mM EDTA and 25% glycerol) in the presence of the protease inhibitors. Following an incubation for 30 min on ice, the solution was centrifuged for 30 min. The supernatants contained the nuclear proteins and were shock-frozen in liquid nitrogen before they were stored at −80° C.

The following protease inhibitors stored at −20° C. were added to buffers A and C: 1.5 μl/ml of peptstatin A (1 mg/ml in 96% ethanol), 1 μl/ml of aprotinin (10 mg/ml in H₂O), 1 μl/ml of leupeptin (10 mg/ml in methanol), 1 μl/ml of DTT (1 M in H₂O), 10 μl/ml of PMSF (17.4 mg/ml in isopropanol).

b) Protein determination: The protein determination was carried out using the processes familiar to the person skilled in the art (see Bradford, 1976). The protein concentration of the nuclear extracts was measured photometrically as an extinction at a wavelength of 595 nm. 1 μl of the sample was mixed, together with 800 μl of H₂O and 200 μl of “BioRad Protein Assay” (BioRad Laboratories, Munich), in the presence of protease inhibitors and incubated for 5 min at room temperature. With the aid of a calibration curve, which was recorded using BSA (1 mg/ml), it was possible to determine the protein content.

c) Sodium dodecylsulfate-polyacrylamide gel electrophoresis (SDS-PAGE): The SDS-PAGE was carried out according to the process familiar to the person skilled in the art. The following solutions and buffers were used for the gel preparation:

1) Separating gel (8%): 8.97 ml of H₂O, 4.8 ml of separating gel buffer (1.5 M tris HCl, pH 8.8, 0.4% SDS), 5 ml of 30% acrylamide/bisacrylamide, 112.5 μl of APS, 22.5 μl of TEMED.

2) Collecting gel (4%): 3.7 ml of H₂₀, 1.5 ml of collecting gel buffer (0.5 M tris HCl, pH 6.8, 0.4% SDS), 0.8 ml of 30% acrylamide/bisacrylamide, 70 μl of APS, 7 μl of TEMED.

In each case, 50 μg of protein sample was diluted 1:2 with buffer C and then 1:2 with “loading dilution” buffer (6.25 ml of 1 M tris HCl pH 6.8, 2 g of SDS, 20 ml of glycerol, a spatula tipful of Bromophenol Blue, to 50 ml of H₂O) and 10% of 1 M β-mercaptoethanol. After denaturation for 5 min at 95° C., the samples and the “Rainbow” molecular weight standard (Amersham, Braunschweig) were applied. The running buffer was composed of 7.56 g of tris base, 36 g of glycine and 2.5 g of SDS, to 2.5 l of H₂O.

d) Protein transfer to membranes: The proteins of the SDS gel were transferred by applying an electric field (100 mA) to a PVDF membrane (Boehringer Mannheim, Mannheim) for approximately 12 hours. As a transfer buffer, the running buffer from c) containing methanol in a final concentration of 20% was used.

e) Antibody labeling: The membrane with the transferred proteins was washed twice for 10 min using PBS (Biowhittaker) before it was incubated for 1 hour with “blocking solution” (1:10) (Boehringer Mannheim) according to the instructions of the manufacturer. The membrane was then incubated with 3 μg/ml of the primary antibody (anti-hdm2 monoclonal antibody IF2, mouse IgG_{2b}) for 2 hours. After washing twice with PBS, 0.1% polyoxyethylenesorbitan monolaurate (Tween 20), the membrane was washed twice with diluted “blocking solution” (1:20). Incubation with a peroxidase-conjugated secondary antibody (goat anti-mouse IgG, 1:10000) for 2 hours followed. The membrane was washed three times for 15 min with PBS, 0.1% of Tween 20, and finally once with PBS.

f) Development: The development of the antibody-labeled membrane was carried out for 1 min in “Solution A”, 1% “Solution B” (Boehringer Mannheim), according to the instructions of the manufacturer. For autoradiography, an X-ray film was placed on the membrane and the labeled proteins were detected by means of their chemiluminescence.

(4) Determination of the peptide binding affinity for HLA-A2.1

A competition test was used in order to determine the binding of the hdm2 peptides to A2.1. EA2 cells were loaded with 0.01 μg of the A2.1-binding peptide p53 264-272 (Theobald et al., 1995) and 3 or 10 μg of hdm2 peptide. The A2.1-binding peptides tyrosinase 369-377 (Wolfel et al., 1994) and the peptide 58-66 of the A/PR/8/34 influenza virus matrix protein M1 (Flu M1 58-66) (Theobald et al., 1995) were used as positive controls, the H-2 K^{b}-binding peptide 52-59 of the vesicular stomatitis virus nucleoprotein (VSV-N 52-59) (Theobald et al., 1995) as a negative control. The A2.1-restricted and p53 264-272-specific CTL (CD8×) A2 264 were investigated at various effector to target cell (E:T) ratios for their lytic activity against peptide-loaded and unloaded EA2 target cells in a 4-hour cytotoxicity test (see chapter B 8) (Theobald et al., 1995). The percentage inhibition of the CTL (CD8 x) A2 264-mediated specific lysis (SL) of p53 264-272-loaded EA2 cells by the test peptides was calculated at an E:T ratio of 1:1 (0.3:1 in the case of hdm2 314-324, 365-375, 402-411 and 419-426) according to the following formula:

$$\text{\% Inhibition} = 100 - \frac{\begin{matrix}[ ( \text{\% SL EA2 plus peptide 264 plus test peptide} - \\ \text{\% SL EA2} )\end{matrix}}{( \text{\% SL EA2 plus peptide 264} - \text{\% SL EA2} ) ]} \times 100$$

(5) Immunization of A2.1-Transgenic Mice and Induction of Peptide-Specific and Alloreactive CTL

For the generation of A2.1-restricted peptide-specific CTL, 8-12 week-old A2.1-transgenic mice were injected subcutaneously in the base of the tail with (50-) 100 μg of the respective test peptide and 120 μg of HBV core 128-140 (an I-A^{b}-binding synthetic T-helper peptide) (Theobald et al., 1995), emulsified in 100 μl of incomplete Freund's adjuvant (IFA; Difco Laboratories, Detroit, USA), (Theobald et al., 1995). After approximately 10 days, the spleen was removed, comminuted and the spleen cell suspension was washed twice (1500 rpm, 5° C., 7 min). The spleen cells were inoculated to 7 Mio/ml/well in a 24-well plate. As stimulator cells, LPS-activated B-cell blasts irradiated with 3000 Rad (¹³²cesium), loaded with 5 μg/ml of the respective test peptide and 10 μg/ml of human β₂-microglobulin, were added thereto to 3 Mio/ml/well after washing twice (Theobald et al., 1995). The LPS blasts were obtained by three-day stimulation of spleen cells (1 Mio/ml) from A2.1-transgenic mice with 25 μg/ml of LPS (Salmonella typhosa) and 7 μg/ml of dextran sulfate (Pharmacia Biotech, Freiburg). The batches of effector and stimulator cells were incubated for 6 days (I° cultures) and subjected to a cytotoxicity test.

Allo-A2.1-reactive I° CTL were generated by incubating spleen cells from CD8-transgenic mice to 7 Mio/ml/well (effector cells) together with irradiated spleen cells from A2.1-transgenic mice to 6 Mio/ml/well (stimulator cells) for 6 days.

(6) Establishment of CTL Lines

Polyclonal peptide-specific CTL lines having specificity for hdm2 81-88 (CTL A2 81 and CD8×A2 K^{b} 81) and for Flu M1 58-66 (CTL CD8×A2 K^{b} Flu M1) were established by weekly restimulation of the effector cells with peptide-loaded stimulator cells. The stimulator cells used were JA2 cells, which were irradiated with 20000 Rad, then loaded in RPMI 1640 (Biowhittaker, Verviers, Belgium) with 5 μg/ml of the respective peptide and 10 μg/ml of human β2-microglobulin for approximately 40 min and finally washed twice. The effector cells were inoculated together with 0.5 Mio JA2 cells and 6 Mio C57BL/6 spleen cells irradiated with 3000 Rad in a total volume of 2 ml/well into a 24-well plate. 2% (v/v) supernatant from the culture medium of Con A-activated spleen cells (TCGF) from Lewis rats was added to the batches (Theobald et al., 1995).

Allo-A2.1-reactive CTL lines were induced by intra-peritoneal immunization of CD8-transgenic mice with 20 Mio JA2 cells/mouse. After three weeks, the spleen cells were isolated and stimulated in vitro (7 Mio/ml/well) with irradiated JA2 cells (0.5 Mio/ml/well) or spleen cells (6 Mio/ml/well) of A2.1-transgenic mice. By repeated weekly in vitro-restimulation with JA2 cells in the presence of irradiated C57BL/6 spleen cells (6 Mio/ml/well) and 2-5% TCGF, allo-A2.1-reactive CTL lines were finally generated.

(7) Extraction and HPLC Fractionation of Natural Peptides and Reconstitution of the CTL Recognition

a) Extraction of natural peptides from MHC class I molecules: Adherent Saos-2/cl 6 cells grew up to a density of approximately 5×10⁷ cells/bottle. The cells were washed twice with HBSS (Biowhittaker, Verviers, Belgium) and MHC class I-bound peptides were extracted by treatment of the cells for 1 min with 5 ml of extraction buffer (0.13 M citric acid, 0.061 M Na₂HPO₄, pH 3.0) (Theobald et al., 1998). After washing twice with RPMI 1640 (Biowhittaker, Verviers, Belgium), the cells were cultured further in cell culture medium (see 2.4). The extracts were centrifuged and the peptide-containing supernatant was frozen. This procedure was repeated every 2 days for 10 days in order to collect peptide extracts from an equivalent of approximately 2×10⁹ Saos-2/cl 6 cells. The extracts were thawed, pooled and loaded on C-18 “spice cartridges” (Analtech Inc., Newark, Del.), which had been washed beforehand with 4 ml of methanol and 4 ml of H₂O. The “cartridges” were washed again with 10 ml of H₂O and the peptides were eluted using 4 ml of aceto-nitrile (contains 0.1% TFA). The peptide-containing eluate was vacuum-dried, resuspended in H₂O and freed of residues by centrifugation. The supernatant was filtered through a Centricon-10 column (Amicon, Beverly, Mass.) and the resulting peptide extract again vacuum-dried (Theobald et al., 1998).

b) HPLC fractionation of natural peptide extracts and reconstitution of the CTL recognition: 0.9 ml of the natural peptide extract resuspended in 0.05% TFA and in each case 1 ml (=100 ng) of the synthetic peptides hdm2 81-88 or hdm2 80-88 were separated on an RP-HPLC SMART system, which was equipped with a μRPC C2/C18 SC 2.1/10 column (Pharmacia Biotech, Uppsala, Sweden), and eluted by means of a gradient, consisting of 20-95% of eluent B (70% acetonitrile in 0.05% TFA) in eluent A (0.05% TFA), in 36 min and a flow rate of 50 μl/min in 2-min fractions to give 100 μl (natural peptide extract and hdm2 81-88) or with a flow rate of 25 μl/min to give 50 μl (hdm2 80-88) (Theobald et al., 1998). HPLC fractions were collected in the range from 30-70 min. ⁵¹Cr-labeled T2-target cells were loaded for 60 min in serum-free RPMI 1640 (Biowhittaker), 5% BSA and 10 μg/ml β₂-microglobulin, with 50 μl of the individual HPLC fractions of the natural peptide extract and with 0.03 μl (hdm2 81-88) or 2.5 μl (hdm2 80-88) of the individual HPLC fractions of the synthetic peptides and sent to a 6-hour cytotoxicity test (Theobald et al., 1998). CTL CD8×A2 K^{b} 81 were employed as the effector cells in an E:T ratio of 20:1.

(8) Cytotoxicity Test

The lytic reactivity of the effector cells against various target cells was checked in a ⁵¹Cr release test (Theobald et al., 1995). T2 and T2A2 K^{b} cells were employed as target cells for peptide titration tests. 1-5 Mio target cells were labeled for 60-90 min with 150 μCi of Na (⁵¹Cr) 04 (1 mCi/ml) (NEN Life Science, Belgium). Before this labeling, 2 μl of peptide solution of differing concentration and 15 μl of FCS (PAA Laboratories, Linz, Austria) or FCS without peptide were added to the cells in peptide titration tests. The labeled target cells were washed four times and the cell count adjusted to 0.1 Mio/ml. Die effector cells were serially diluted 1:3 with the cell culture medium and inoculated to 0.1 ml/well in 96-well plates. Altogether, five different E:T ratios were tested. 0.1 ml/well of the target cell suspension was then added to the effector cells and the batches were incubated for 4-6 hours. The cells were then centrifuged off (1300 rpm, 5° C., 9 min), the supernatant (0.1 ml/well) was taken off and the ⁵¹Cr release was measured using a gamma-“counter” (Canberra Packard, Dreieich). The percentage specific lysis (SL) was calculated according to the following formula:

$$\frac{( \text{experimental Cr release} - \text{spontaneous Cr release} )}{( \text{maximum Cr release} - \text{spontaneous Cr-release} )} \times 100 = \text{\% SL}$$

(experimental Cr release - spontaneous Cr release)×100=% SL (maximum Cr release - spontaneous Cr-release)

The maximum ⁵¹Cr release corresponded to the total ⁵¹Cr incorporation by the target cells, the spontaneous ⁵¹Cr release corresponded to the target cell lysis in the absence of effector cells and was as a rule less than 10% of the maximum ⁵¹Cr release. The values for spontaneous and maximum lyses were averaged from four batches in each case, those for experimental lyses from two batches.

### C) EXAMPLES

### Example 1

### Experimental Obtainment of the Oligopeptide hdm2 81-88

(1.1) Selection of Potentially A2.1-Binding hdm2 Peptides

By means of the known amino acid sequence of the hdm2 oncoprotein, 8mers, 9mers, 1Omers and 11mers were determined, which are subsequences of this hdm2 polypeptide and fulfill the following criteria:

1.) They have as “primary anchor amino acids”, that is amino acids within the peptide which interact with residues of the binding pocket of the MHC class I molecule and in the case of endogenously processed and in the context of MHC class I molecules presented peptides are situated in position 2 and at the C-terminus of the epitope, in position 2 classically the amino acids L, M, I, V or T, and nonclassically the amino acids A, Q or K and at the C-terminus classically the amino acids V, L or I and nonclassically the amino acids A, M or T (Theobald et al., 1995).

2.) The hdm2 peptides should if possible not be homologous to the corresponding mdm2 peptides of the mouse.

3.) The 9mers should possess as high a “score” as possible, which is based on binding data of synthetic peptides (Parker et al., 1994).

Altogether, 51 hdm2 peptides were selected (see FIG. 1).

(1.2) Binding of Selected Synthetic hdm2 Peptides to A2.1

The hdm2 peptides selected according to (1.1) by means of their theoretical binding strength were investigated for their actual binding affinity for A2.1. For this, in a competitive binding test, which is described in greater detail in the publication of Theobald et al. (1995), the ability of the hdm2 peptides to inhibit the A2.1 binding of the competing synthetic peptide p53 264-272 was tested functionally. This inhibition was measured by means of the decrease in the lysis of EA2 cells, which were loaded with p53 264-272 peptide and the individual hdm2 test peptide, mediated by an A2.1-restricted p53 264-272-specific CTL line. The binding results are presented in summarized form in FIG. 1. The peptide tyrosinase 369-377, which was used as a positive control, showed the strongest inhibition and thus binding to A2.1 (cf. Wolfel et al., 1994), and achieved 100% inhibition both at 3 and at 10 μg, while the H-2 K^{b}-binding peptide VSV-N 52-59 (Theobald et al., 1995), as a negative control, showed no A2.1-binding activity at all. The hdm2 peptides were divided into 4 groups according to their binding strength. Of altogether 51 peptides tested, 12 had a high binding activity (at least 85% inhibition at 10 μg of test peptide), 16 a medium activity (50-84% inhibition), 13 a weak activity (10-49%) and 10 no binding activity (<10% or low-dose dependence of the inhibition). The strongest-binding hdm2 peptides were 80-88, 81-88, 48-57 and 33-41 at 10 μg with in each case 100% inhibition of the binding of the competing peptide p53 264-272. The inhibition of the binding was dose-dependent, since for all A2.1-binding peptides the inhibition values at 10 μg were markedly above those at 3 μg. Altogether, 55% of all peptides selected showed a strong or intermediate A2.1 binding, only 20% were not able to bind to A2.1.

### Example 2

### Experimental Demonstration of the Suitability of the hdm2 81-88 Oligopeptide for the Production of a Specific, CTL-Mediated Immunogenicity

(2.1) Immunogenicity of A2.1-Binding Synthetic hdm2 Peptides in A2.1-Transgenic Mice

An obstacle in the recognition of human MHC class I molecules by mouse T cells is the inability of mouse CD8, to interact with HLA molecules such as A2.1. For the circumvention or removal of this obstacle, two strategies were used. One strategy consisted in the construction of the chimeric molecule A2.1/K^{b} (A2 K^{b}), which is composed of the human α1 and α2 domains of A2.1 and of the α3 domain of mouse K^{b}, which is essential for the interaction with CD8. CTL induced in A2K^{b}-transgenic mice with restriction for the A2K^{b} transgene recognize the same peptide antigens which are also immunogenic in A2.1-positive humans. The other strategy for the amplification of the A2.1-restricted response consisted in the production of a double transgenic mouse “CD8×A2.1/K^{b}” by crossing an A2 K^{b}-transgenic mouse with an huCD8α/β transgenic mouse. The expression of the α- and β-chain of the huCD8 molecule enables the generated CTL to interact with the α3 domain of the A2.1 molecule of human cells.

A2K^{b}- and CD8×A2K^{b} transgenic mice were immunized with the strongly or intermediately binding peptides obtained according to example 1 (see FIG. 1) in order to obtain hdm2 peptide-reactive CTL. 9 to 11 days after the immunization, spleen cells of the mice concerned were stimulated in vitro with peptide-loaded syngeneic LPS blasts and 6 days thereafter investigated in a cytotoxicity test for an A2.1-restricted peptide-specific CTL response. The results are shown in summarized form in FIG. 2. For the positive control Flu M1 58-66, the induction of A2.1-restricted CTL was already known (Theobald et al., 1995). An A2.1-restricted and peptide-specific CTL response was demonstrated for the strongly binding peptides hdm2 81-88, 33-41 and 80-88 and for the intermediately binding peptide hdm2 101-110. The level of the lysis was dependent on the E:T ratio. The CTL were peptide-specific, since they lyzed cells loaded with the corresponding peptide, but not cells which were loaded with irrelevant A2.1-binding peptides (data not shown).

The immunogenicity of the peptide hdm2 80-88 was probably based on a contamination with hdm2 81-88, since after immunizations with hdm2 80-88 carried out independently, the CTL recognition decreased with increasing purity of the peptide. The contamination could also be demonstrated by mass spectrometry (data not shown). CTL induced by hdm2 81-88 were A2.1-restricted, since A2.1-negative EL4 cells (H-2^{b}) of the mouse loaded with the corresponding peptide were not recognized (FIG. 3).

(2.2) hdm2 81-88-Specific CTL: A2.1 Restriction, Peptide Specificity and Efficiency of the Peptide Recognition

CTL which were A2.1-restricted and specific for hdm2 81-88 were investigated in greater detail below. Since up to this point in time in the study only hdm2 81-88-specific CTL lines generated from A2K^{b}-transgenic mice existed, A2.1 and CD8×A2 K^{b} transgenic mice were immunized with hdm2 81-88 with the intention of obtaining CTL having higher avidity.

After immunization of A2.1- and CD8×A2K^{b}-transgenic mice with hdm2 81-88, the spleen cells were stimulated with peptide-loaded LPS blasts from A2.1-transgenic mice (I° culture) and tested 6 days later in the cytotoxicity test against T2 target cells, incubated at different concentrations of synthetic peptide hdm2 81-88 (FIG. 4A). The I° CTL cultures A2.1 (A2) and CD8×A2 K^{b} 81 differed in their peptide recognition efficiency by the factor 5. The half-maximal lysis of the target cells by I° CTL A2 81 was at a peptide concentration of 0.95 nM in comparison with 0.2 nM by I° CTL CD8×A2 K^{b} 81. From the difference in the peptide recognition efficiency, it can be derived that I° CTL CD8×A2 K^{b} 81 possess a higher avidity than I° CTL A2 81. The absolute maximal lysis in the case of I° CTL CD8×A2 K^{b} 81 at 100% was also significantly higher than in the case of I° CTL A2 81 at 62%. This difference in the avidity of hdm2-reactive T cells is also reflected in the recognition of endogenously presented hdm2 81-88 peptide (FIGS. 4B and C). While I° CTL CD8×A2 K^{b} 81 lyzed the hdm2-overexpressing and A2.1-positive transfectant Saos-2/cl 6 at an E:T ratio of 30:1 to 42%, I° CTL recognized A2 81 Saos-2/cl 6 only to 23%. The osteosarcoma cell line Saos-2, which expresses no detectable hdm2 protein and was therefore used as a negative control, was not recognized by I° CTL (for this see also FIG. 6).

These results show that after a single immunization of A2.1- and CD8×A2K^{b} transgenic mice and a single in vitro stimulation with the hdm2 81-88 peptide, highly avid CTL were induced which recognized endogenously presented peptide. For the recognition of hdm2 transfectants see example 4.

By repeated restimulation of I° CTL from A2.1- and CD8×A2K^{b}-transgenic mice with peptide-loaded stimulator cells, stable CTL lines having specificity for hdm2 81-88 were generated. FIG. 5A shows the efficiency of the recognition of synthetic hdm2 81-88 by both CTL lines at an E:T ratio of 10:1. The avidity of the CTL line A2 81 for the I° CTL increased by more than one log stage, since the half-maximal lysis of the target cells was achieved at a peptide concentration of 0.069 nM. The lytic activity by CTL CD8×A2K⁶ 81 was, at 0.036 nM, half-maximal, which corresponded to an increase in the sensitivity by the factor 5. The observed increase in the avidity of the CTL lines is to be attributed to the expression of highly avid hdm2-reactive CTL. Both CTL lines were peptide-specific, since T2 cells loaded with hdm2 81-88 were lyzed efficiently, while T2 target cells which were unloaded or loaded with the irrelevant peptide Flu M1 58-66 were not recognized (FIGS. 5B and C). Flu M1 58-66-presenting T2 cells were lyzed, however, by a CD8×A2 K^{b} T cell population having specificity for Flu M1 58-66 (without Fig.). Moreover, the hdm2 81-88-reactive CTL lines were A2.1-restricted, since with A2.1-negative and hdm2 81-88-loaded EL4 cells (H-2^{b}) of the mouse no lytic activity at all was to be observed.

In the end result, highly avid A2.1-restricted CTL-populations having specificity for hdm2 81-88 were generated.

### Example 3

### Characterization of hdm2-Transfected Cell Lines

In order to determine whether the peptide hdm2 81-88 is actually endogenously processed and is presented in the context of A2.1 molecules of hdm2-overexpressing tumor cells, various hdm2-negative (Saos-2, EL4) or hdm2-low-expressing (SW 480) tumor cell lines were transfected with the hdm2 gene (Oliner et al., 1992). The recognition of the resulting hdm2-overexpressing transfectants by hdm2 81-88-specific CTL is an index of the endogenous production of the peptide hdm2 81-88. For the transfection with the hdm2 gene, the tumor cell lines Saos-2, SW480 and EL4 (H-2^{b}) were selected. Saos-2 is a p53-deficient and A2.1-positive osteosarcoma line and particularly suitable for the hdm2 transfection, since p53 is a transcription activator for the hdm2 gene and thus no significant endogenously expression of hdm2 is to be expected in Saos-2. SW480 is an A2.1-positive colon carcinoma line and expresses small amounts of hdm2 protein. EL4 is an A2.1-negative thymoma line of the mouse lacking hdm2 expression.

By lipofection of the cell line Saos-2 with the plasmid pCHDMIA, which codes for the hdm2 protein and the neomycin resistance (FIG. 26), transfectants were generated which constitutively overexpressed the hdm2 under the control of the CMV promoter. Nuclear extracts were prepared from the cells, since hdm2 is mainly located in the nucleus. The extracts were separated by gel electrophoresis, transferred to membranes, labeled with anti-hdm2 antibody and finally visualized via chemi-luminescence. The Western blot according to FIG. 6 shows the hdm2 protein expression of the hdm2 transfectants Saos-2/cl 5 and Saos-2/cl 6. While at 90 kDa a clear and at 75 kDa a weak protein band is to be recognized (arrows), the parental Saos-2 cells as expected expressed no hdm2 protein. The 90 kDa protein is the full-length hdm2 product of 491 amino acids (cf. Oliner et al., 1992), while the 75 kDa product was translated from an hdm2-mRNA “splice” variant having a deletion of the bases 158-667 (Sigalas et al., 1996). The pre-B ALL cell line EU-3 used as a positive control (Zhou et al., 1995) showed a very strong expression both of the 90 kDa and of the 75 kDa protein.

For an effective presentation of the hdm2 peptides, a prerequisite is, inter alia, an adequate expression of A2. The flow cytometry analysis of the hdm2 transfectants showed a comparable A2 expression of Saos-2/cl 5 and 6, which was only insignificantly stronger than that of the parental Saos-2 cells (FIG. 7).

EL4 cells of the mouse were cotransfected with the plasmid pSV₂A2 (FIG. 27), which codes for the A2.1 molecule (Theobald et al., 1995), and pCHDMIA by means of electroporation. FIG. 6 shows the significant expression of the 90 kDa full-length hdm2 protein by the A2.1-positive transfectant EA2/cl 13 in contrast to hdm2-negative EA2 cells. Both transfectants were comparable in their A2.1 expression (data not shown). Moreover, the colon carcinoma line SW480, which only expressed a little hdm2, was lipofected with pCHDMIA, where, however, initially no significant difference in the hdm2 expression of the resulting clone SW480/cl 2 and the parental cells in the Western blot was to be observed (data not shown).

### Example 4

### Recognition of hdm2 Transfectants by hdm2 81-88-Specific CTL

For checking the natural processing and A2.1 presentation of the peptide hdm2 81-88, the hdm2 transfectants were tested for their recognition by A2.1-restricted hdm2 81-88-specific CTL. The Saos-2 transfectants Saos-2/cl 5 and 6 were efficiently lyzed by the hdm2-reactive CTL A2 and CD8×A2 K^{b} 81, while the parental Saos-2 line was not recognized and consequently not lyzed (FIG. 8). It was possible for the lysis of the transfectants to be inhibited by the anti-A2.1 monoclonal antibody PA2.1, which is further proof for the A2.1 restriction of the hdm2-reactive CTL. As already explained, CTL CD8×A2 K^{b} 81 also showed a higher lysis of the target cells than CTL A2 81 in the endogenous recognition, possibly due to CD8-mediated increase in the avidity. The CTL line CD8 allo A2 was used as a positive control. Both the hdm2 transfectants and the parental cells were lyzed by the allo-A2.1-reactive effector cells (FIG. 8). Since these alloreactive CTL were peptide specific, i.e. recognized A2.1 molecules only in context with (processed) self-peptides (but not signal peptides) (results not shown), in this way possible deficits, e.g. in the transport system of the investigated cells, were able to be quasi-excluded. The A2.1-restricted CTL line CD8×A2 K^{b} Flu M1, which lyzed none of the tested cell lines, functioned as a negative control (FIG. 8).

The recognition of the hdm2 transfectants EA2/cl 13 and SW480/cl 2 is shown in FIGS. 9 and 10. The lysis of these target cells by hdm2 81-88-specific CTL was less efficient in comparison with Saos-2/cl 5 and 6, but blockable. The parental cell lines were, as expected, not recognized by the hdm2-reactive CTL (FIGS. 9 and 10). Allo-A2.1-reactive CTL lyzed all, Flu M1-specific CTL but none of the target cells offered (FIGS. 9 and 10). Although in the case of SW480/cl 2, an hdm2 overexpression in the Western blot was not detectable in comparison with SW480, SW480/cl 2 was significantly lyzed by CTL CD8×A2 K^{b} 81, which could point to a comparatively higher sensitivity of the cytotoxicity test. Moreover, it is conceivable that the number of the specific peptide-MHC complexes of SW480/cl 2 cells is greater than that of SW480 cells, since SW480/cl 2 was more susceptible to allo-A2.1-reactive T cells than the parental cell line (FIG. 10).

All hdm2 transfectants used in these experiments were transfected with the pCHDMIA expression plasmid (FIG. 26). This codes for the hdm2 protein and additionally for the neomycin resistance, which functions as a selection marker. The presumption was obvious that the peptide hdm2 81-88 was processed endogenously and was presented in the context of A2.1 and thus represented the epitope for the hdm2-reactive CTL. Since these CTL were populations, however, the presence of T-cell subpopulations having specificity for peptides which were processed from the neomycin resistance was not to be excluded, especially as the restimulation of the CTL took place with neomycin-resistant transfectants. However, the absent recognition of the EA2 and EA2 K^{b} controls which, like the hdm2 transfectants too, express neomycin resistance, is a point against a lysis of the hdm2 transfectants by potential subpopulations having specificity for the neomycin resistance. Moreover, for example, with CTL clone 3, which had been isolated from the CTL population CD8×A2 K^{b} 81, comparable cytotoxicity data with the hdm2 transfectants as target cells were obtained (data not shown). Since a CTL clone in general is strictly peptide-specific, the observed lysis of the hdm2 transfectants is to be attributed to hdm2 81-88-specific recognition.

A further clear index for hdm2 81-88 as a T-cell epitope was the lysis of various hdm2-overexpressing tumor cells (see Example 6), while in contrast thereto Saos-2 cells showed no detectable hdm2 expression and were not recognized. Accordingly, the hdm2 81-88 oligopeptide is also not an epitope of other processed self-proteins.

The results shown here point to the fact that hdm2 81-88 peptide is actually processed endogenously and is presented in the context of A2.1.

### Example 5

### Demonstration of the Identity of the Synthetic Peptide hdm2 81-88 with the Natural A2.1-Presented hdm2-CTL Epitope

In order to demonstrate that the natural A2.1-presented CTL epitope are identical for the hdm2 81-88-specific CTL and the synthetic peptide hdm2 81-88, natural peptides were extracted from MHC class I molecules by acid treatment (Lustgarten et al., 1997). The concentrated and purified peptide extracts and the synthetic hdm2 81-88 peptide were then further purified by means of HPLC. The resulting, individual natural or synthetic HPLC fractions were loaded on T2 cells and their recognition was tested by hdm2 81-88-specific CTL (Lustgarten et al., 1997). FIG. 11 shows the recognition of the respective HPLC fractions of the natural peptide extract of Saos-2/cl 6 as a function of their retention time by means of CTL CD8×A2 K^{b} 81.

CTL lysis was reconstituted with HPLC fraction 21 of the natural peptide extract. Comparable results were obtained with CTL A2 81 and CTL clone 3 CD8×A2 K^{b} 81. CTL lysis of the HPLC-fractionated synthetic peptide hdm2 81-88 was reconstituted by fraction 21, which had an identical retention time in comparison with the antigenic fraction 21 of the natural peptide extract (FIG. 11). In the HPLC profile, the synthetic peptide also eluted in fraction 21, which proves that the observed lytic activity is to be attributed to the specific recognition of hdm2 81-88 alone.

In order to exclude that the recognized T-cell epitope was represented by the peptide hdm2 80-88, and the recognition was based on a cross-reaction, the synthetic and to 90% pure peptide hdm2 80-88 was further purified by means of HPLC. The T-cell recognition of the resulting HPLC fractions showed two “peaks”, the first in fraction 21 with a retention time which is virtually identical in comparison with hdm2 81-88, the second in fraction 23 (data not shown). While the first “peak” was based on a contamination of hdm2 80-88 with the synthesis breakdown product hdm2 81-88—as the mass spectrometric analysis confirmed —the second “peak” was to be attributed to the cross-reactivity of the hdm2 81-88-specific CTL with hdm2 80-88. In the HPLC fractions of the natural peptide extract, however, lysis occurred only in fraction 21, which possessed a retention time identical to the recognized fraction of the synthetic peptide hdm2 81-88. No lysis was detectable in fraction 23. On account of the cross-reactivity, however, lysis must also have taken place in fraction 23 if hdm2 80-88 was naturally presented.

These results point to the fact that the naturally processed and A2.1-presented CTL epitope is actually the peptide hdm2 81-88.

### Example 6

### Use of hdm2 81-88-Specific CTL for the Specific Recognition and Lysis of Human Tumor Cells

(6.1) hdm2 Protein Expression of Human Tumor Cell Lines

For the demonstration that hdm2 81-88-specific CTL not only efficiently lyze hdm2 transfectants but also non-transfected A2-positive tumor cell lines, ALL cell lines were employed for which the overexpression of hdm2-mRNA, but not of hdm2 protein, is known (cf. Zhou et al., 1995). Since in addition to the overexpression of hdm2 protein the presence of A2 is a prerequisite for the CTL recognition, these cell lines were first analyzed by flow cytometry. Of 13 investigated ALL cell lines, two were A2-positive and one A2.24-positive (data not shown). Of these two ALL lines, and three further A2-positive ALL, lymphoma and plasmocytoma lines, Western blots were carried out, since for peptide presentation, in the final analysis, among other things the overexpression of hdm2 protein, but not of hdm2-mRNA, is of importance. In FIG. 12, the hdm2 protein expression of various A2-positive human tumor cell lines is shown. All investigated leukemia lines and one lymphoma and one plasmocytoma line overexpressed the 90 kDa full-length hdm2 product. The 75 kDa “splice” variant of hdm2 was likewise produced in recognizable amounts by these cell lines.

The hdm2 protein expression of three A2-negative ALL cell lines is shown in FIG. 13. Here too, the data for the protein expression agreed with those for the mRNA expression (Zhou et al., 1995), so that in the case of all ALL cell lines investigated here a post-transcriptional mechanism obviously does not form the basis of the hdm2 overexpression. Exceptions are the pre-B ALL cell lines EU-6 and EU-8, for which a weak or absent hdm2-mRNA expression has been described (Zhou et al., 1995). These cell lines, however, show a strong or moderate hdm2 protein expression in the Western blot (data not shown).

(6.2) Recognition of hdm2-Overexpressing A2-Positive Tumor Cell Lines by hdm2 81-88-Specific CTL

The hdm2 protein overexpressing and A2-positive tumor cell lines from FIG. 12 were used below as target cells for hdm2 81-88-specific CTL in order to demonstrate that not only hdm2-transfected, but also non-transfected tumor cells are efficiently lyzed.

Saos-2/143 cells, which were transfected with mutated p53 (Theobald et al., 1995), but not with hdm2, were recognized in contrast to the parental Saos-2 cells of CTL CD8×A2 K^{b} 81 (FIG. 14). It was possible to increase the lysis by 20-hour pretreatment of the target cells with IFN-γ (20 ng/ml) and to inhibit it by addition of PA2.1. The recognition of untreated Saos-2 and Saos-2/143 cells by the allo-A2-reactive CTL used as a positive control was comparable, the recognition of IFN-γ-treated Saos-2/143 cells was better than the untreated (FIG. 14). The reason for the improved lysis of IFN-γ-treated cells is, inter alia, the increased expression of MHC-peptide complexes and adhesion molecules. With the Flu M1 58-66-specific negative control CTL CD8×A2 K^{b} Flu M1, however, no lysis was to be observed.

The recognition of hdm2-overexpressing and A2-positive B-ALL cell lines by hdm2-reactive CTL was investigated below. The cell line EU-3 was recognized by CTL A2 and CD8×A2 K^{b} 81, CTL CD8×A2 K^{b} 81 achieving 100% lysis at an E:T ratio of 30:1 (FIG. 15). The lysis by both CTL lines was completely blocked by PA2.1. In these experiments, allo-A2.1-reactive T cells, which were primarily generated in vitro and therefore showed a lower efficiency of recognition than the CTL line CD8 allo A2, functioned as a positive control. Contrary to EU-3, no lytic activity was found on the part of the CTL line CD8×A2 K^{b} Flu M1. Comparable results were achieved with the pre-B ALL cell lines UoC-B11 and BV173 as target cells for CTL CD8×A2 K^{b} 81 (FIG. 16). At an E:T ratio of only 0.3:1, both cell lines were lyzed to more than 50%. Here too, it was possible with PA2.1 to achieve a complete inhibition of the recognition. Although the lysis of UoC-B11 by allo-reactive CTL was at least twice that of BV173, this did not have an effect—in the case of comparable hdm2 expression (see FIG. 12)—on the level of the hdm2-specific recognition (FIG. 16). Obviously, for CTL CD8×A2 K^{b} 81 the amount of peptide:MHC class I complexes was not limiting. These cell lines were not susceptible to the Flu M1-specific T cells. The cell lines OPM-2 (plasmocytoma) and U-937 (histiocytic lymphoma) were likewise lyzed selectively (FIG. 17).

These findings showed that CTL having specificity for hdm2 81-88 A2-positive tumor cells which endogenously overexpressed hdm2, recognized and lyzed specifically, 2-restrictedly and efficiently.

(6.3) A2-Negative hdm2-Overexpressing Tumor Cell Lines are not Lyzed by A2-Restricted hdm2-Reactive CTL

For checking the recognition of the A2-positive tumor cell lines by hdm2 81-88-specific CTL, cell lines were used which admittedly overexpressed hdm2 (see FIG. 13), but showed no A2 phenotype in the flow cytometric analysis (data not shown). The pre-B ALL cell lines UoC-B4, EU-1 and SUP-B15 were not lyzed by CTL CD8×A2 K^{b} 81 and allo-A2.1-reactive CTL (FIG. 18). A2-positive EU-3 cells were efficiently recognized on the part of these CTL lines. No lysis was to be observed, however, with Flu M1-specific CTL.

These experiments and their results demonstrate that the recognition of hdm2-overexpressing tumor cells takes place A2-restrictedly, and that it can be excluded that the observed lysis of A2-positive tumor cells was mediated by natural or lymphokine-activated killer cells.

### Example 7

### Use of hdm2 81-88 Specific CTL for the Selective Recognition and Lysis of Human Tumor Cells

(7.1) hdm2 Protein Expression of Transformed, Activated or Resting Cells of Lymphohemopoietic Origin

For a potential, hdm2-specific CTL-mediated immuno-therapy, it is desirable that normal cells are not lyzed. The hdm2 oncoprotein is overexpressed in malignant hematological diseases (see Example 6, (6.1)) and, as is known, is also expressed by some normal cells, among them also lymphohemopoietic cells.

In FIG. 19, the hdm2 protein expression of the lymphohemopoietic cells of differing transformation and activation state is shown. The EBV-transformed lymphoblastoid cell line (LCL) LG-2 showed a very strong expression of the hdm2 protein. PHA- and Con A-transformed blasts expressed significantly lower, but still substantial amounts of hdm2 protein. For comparison, the hdm2 protein expression of nontransformed normal cells was juxtaposed to these transformed B- and T-cell blasts. In the case of the antigen-activated tyrosinase 369-377-specific T-cell clone IVSB (Wolfel et al., 1994), no hdm2 protein was detected in the Western blot (FIG. 19). Additionally, the hdm2 protein expression of resting T cells, B cells and PBMC was investigated. In these cells too, no hdm2 protein was detected.

(7.2) Cytolytic Reactivity of hdm2 81-88-Specific CTL to Transformed, Activated or Resting Cells of Lymphohemopoietic Origin

Transformed and nontransformed lymphohemopoietic cells were employed below as target cells for A2-restricted CTL having specificity for hdm2 81-88. The A2-positive EBV-transformed LCL LG-2 and A2-positive PHA- and Con A-transformed blasts were efficiently lyzed by CTL CD8×A2 K^{b} 81 (FIG. 20). These cytotoxicity data are in accord with the data for the hdm2 protein expression (see FIG. 19). The lysis was A2-restricted, since it was almost completely possible to block it with the anti-A2.1 monoclonal antibody PA2.1. The allo-A2.1-reactive CTL used as a positive control recognized all 3 cell types, however, with the Flu M1-specific CTL functioning as a negative control, no lysis was to be observed.

Fully developed dendritic cells (DC) express MHC class I and II, costimulatory and adhesion molecules and are therefore particularly suitable as antigen-presenting cells for CTL. These mature DC were not sufficiently recognized by CTL A2 and CD8×A2 K^{b} 81 (FIG. 21). After loading the DC with exogenous peptide hdm2 81-88, CTL lysis was reconstituted. Since, moreover, a lytic activity on the part of allo-A2.1-reactive CTL took place, it was possible to exclude potential deficits in the A2 expression. No recognition by Flu M1-specific CTL took place.

The results point to the fact that mature DC express no detectable hdm2 protein.

In contrast to transformed EBV-LCL, PHA- and Con A-blasts, mature DC and antigen-activated T cells are not transformed, but specifically activated. As an example of antigen-activated CTL, the tyrosinase-specific and A2.1-positive clone IVSB (Wolfel et al., 1994) was employed as a target cell for CTL having specificity for hdm2 81-88 (FIG. 22). Just as in the case of the DC, no sufficient lysis was recognizable and it was possible to reconstitute the CTL lysis by means of exogenous peptide hdm2 81-88. The allo-A2.1-reactive peptide-specific CTL CD8 allo A2 indicated, with the lysis of IVSB, not only an adequate A2 expression of the target cells, but on account of their strict peptide dependence (data not shown), also functional antigen processing and presentation. CTL CD8 allo A2 in fact do not recognize the A2 molecules per se, but exclusively in the context with endogenously processed cellular self-peptides.

In order to exclude resting cells of lymphohemopoietic origin being activated by the isolation method, resting T and B cells were tested for their sensitivity to hdm2-reactive CTL. Neither T cells (FIG. 23) nor B cells (FIG. 24) were recognized by hdm2-reactive CTL. Likewise, no lytic activity against resting PBMC was to be observed (FIG. 25). In the case of all 3 cell types, it was possible for CTL recognition to be reconstituted by exogenous peptide hdm2 81-88, which confirmed an adequate A2 expression. The ability of the target cells to process and present endogenous self-peptides was checked using their lysis by the peptide-dependent CTL CD8 allo A2. No lytic activity was found on the part of the CTL CD8×A2 K^{b} Flu M1.

### Example 8

### Preparation of A2.1-Restricted T-Cell Receptors which are Specific for the Oligopeptide hdm2 81-88 According to the Invention

A2.1-transgenic mice are immunized with the oligopeptide hdm2 81-88 according to the invention. After 10 days, the spleen is removed. The spleen cells are stimulated in vitro using previously prepared, A2.1-positive antigen-presenting cells, which are loaded with the oligopeptide according to the invention. The preparation of the these 2.1-positive antigen-presenting cells is carried out using the techniques which are known in the prior art and familiar to the person skilled in the art. After culture for a number of weeks, the T cells are checked for their peptide and tumor recognition, peptide specificity and A2.1 restriction. After successful testing, the T-cell line is cloned. The resulting T-cell clones are again tested with respect to peptide and tumor recognition, peptide specificity and A2.1 restriction.

The total mRNA of a T-cell clone having a positive test result is prepared. By means of RT-PCR, the T-cell receptor α- and β-chains are amplified. The respective chains are first cloned into bacterial plasmids and sequenced. The chains are partially humanized by replacing the constant mouse regions by the homologous human regions. The cloning of the resulting constructs into suitable retroviral vectors is then carried out. Peripheral blood lymphocytes of an A2.1-positive cancer patient whose tumor or leukemia cells overexpress hdm2 protein are removed, transduced in vitro using the vectors for the α- and β-chain of the T-cell receptor and the gene expression is investigated at the protein level. T-cell receptor-expressing T lymphocytes are analyzed for their ability to lyze tumor cells. After successful testing, the gene-modified lymphocytes are transfused into the patient and should bring about the destruction of the degenerated cells and thus recovery.

### LIST OF REFERENCES

Bradford M. M. (1976). A rapid and sensitive method for the quantitation of microgram quantities of protein utilizing the principle of protein-dye binding. Anal. Biochem. 72, 248-254.

Dittmer D., Pati S., Zambetti G., Chu S., Teresky A. K., Moore M., Finlay C., Levine A. J. (1993). Gain of functions mutations in p53. Nat. Genet. 4, 42-46.

Irwin M. J., Heath W. R., Sherman L. A. (1989). Species-restricted interactions between CD8 and the a3 domain of class I influence the magnitude of the xenogeneic response. J. Exp. Med. 179, 1091-1101.

Jonuleit H., Kuhn U., Muller G., Steinbrink K., Paragnik L., Schnitt E., Knop J., Enk A. H. (1997). Pro-inflammatory cytokines and prostaglandins induce maturation of potent immunostimulatory dendritic cells under fetal calf serum-free conditions. Eur. J. Immunol. 27, 3135-3142.

Lustgarten J., Theobald M., Labadie C., LaFace D., Peterson P., Disis M. L., Cheever M. A., Sherman L. A. (1997). Identification of Her-2/neu CTL epitopes using double transgenic mice expressing HLA-A2.1 and human CD8. Human Immunol. 52, 109-118.

Meziere, C., Viguier M., Dumortier H., Lo-Man R., Leclerc C., Guillet J. G., Briand J. P., Muller S. (1997), In vivo T helper cell response to retro-inverso peptidomimetics. J. Immunol. 159 (7), 3230-3237.

Oliner J. D., Kinzler K. W., Meltzer P. S., George D. L., Vogelstein B. (1992). Amplification of a gene encoding a p53-associated protein in human sarcomas. Nature 358, 80-83.

Parker K. C., Bednarek M. A., Coligan J. E. (1994). Scheme for ranking potential HLA-A2 binding peptides based on independent binding of individual peptide side-chains. J. Immunol. 152, 163-175.

Salter R. D., Cresswell P. (1986). Impaired assembly and transport of HLA-A and -B antigens in a mutant TxB cell hybrid. EMBO J. 5, 943-949.

Sigalas I., Calvert H. A., Anderson J. J., Neal D. E., Lunec J. (1996). Alternatively spliced mdm2 transcripts with loss of p53 binding domain sequences: Transforming ability and frequent detection in human cancers. Nat. Med. 2, 912-917.

Theobald M., Biggs J., Dittmer D., Levine A. J., Sherman L. A. (1995). Targeting p53 as a general tumor antigen. Proc. Natl. Acad. Sci. USA 92, 11993-11997.

Theobald M., Ruppert T., Kuckelkorn U., Hernandez J., Häuβler A., Antunes Ferreira E., Liewer U., Biggs J., Levine A. J., Huber C., Koszinowski U. H., Kloetzel P.-M., Sherman L. A (1998). The sequence alteration associated with a mutational hotspot in p53 protects cells from lysis by cytotoxic T lymphocytes specific for a flanking peptide epitope. J. Exp. Med. 188, 1017-1028.

Wölfel T., Van Pel A., Brichard V., Schneider J., Seliger B., Meyer zum Buschenfelde K. H., Boon T. (1994). Two tyrosinase nonapeptides recognized on HLA-A2 melanomas by autologous cytolytic T lymphocytes. Eur. J. Immunol. 24, 759-764.

Wu X., Bayle H., Olson D., Levine A. J. (1993). The p53-mdm-2 autoregulatory feedback loop. Genes and Dev. 7, 1126-1132.

Zhou M., Yeager A. M., Smith S. D., Findley H. W. (1995). Overexpression of the MDM2 gene by childhood acute lymphoblastic leukemia cells expressing the wild-type p53 gene. Blood 85, 1608-1614.

1. A universal tumor-associated oligopeptide which is recognized as a peptide antigen by CD8-positive cytotoxic T lymphocytes (CTL) and produces a CTL-induced lysis and/or apoptosis of tumor or leukemia cells,
characterized in that
the oligopeptide (a) has the amino acid sequence LLGDLFGV, which corresponds to the amino acid positions 81 to 88 of the human mdm2 (=hdm2) proto-oncoprotein, or an amino acid sequence derivable by amino acid substitution, deletion, insertion, addition, inversion and/or by chemical or physical modification of one or more amino acids thereof, which is a functional equivalent of the amino acid sequence LLGDLFGV, in that it (b) is an epitope for CD8-positive CTL, and in that (c) it is suitable for inducing a restricted immune response of CD8-positive CTL to human leukocyte antigen (HLA) of the molecular group “MHC class I”, allele variant “A2”, in particular subtype A2.1, agalnst tumor and leukemia cells.

2. A retro-inverse peptide or pseudopeptide, characterized in that it corresponds to an oligopeptide as claimed in claim 1, in which instead of the —CO—NH— peptide bonds —NH—CO— bonds or other nonpeptide bonds are formed.

3. A polynucleotide having a nucleotide sequence which codes at least for an oligopeptide as claimed in claim 1.

4. The use of an oligopeptide as claimed in claim 1 and/or of a retro-inverse peptide or pseudopeptide as claimed in claim 2 and/or of a polynucleotide as claimed in claim 3 for the production of diagnostics and/or therapeutics and/or prophylactics for the detection and/or the influencing and/or generation and/or expansion and/or control of the activation and functional state of T cells, in particular CD8-positive cytotoxic T lymphocytes.

5. A reagent for the in-vivo- or in-vitro activation of T cells, in particular CD8-positive cytotoxic T lymphocytes, characterized in that the reagent is prepared using at least one oligopeptide as claimed in claim 1 and/or a retro-inverse peptide or pseudopeptide as claimed in claim 2 and/or a polynucleotide as claimed in claim 3.

6. A recombinant DNA or RNA vector molecule which contains at least one or more polynucleotide(s) as claimed in claim 3 and which is expressible in cells of autologous, allogenic, xenogenic or microbiological origin.

7. A host cell which contains a polynucleotide as claimed in claim 3 or a vector molecule as claimed in claim 6.

8. The use of at least one oligopeptide as claimed in claim 1 and/or of a retro-inverse peptide or pseudopeptide as claimed in claim 2 for the preparation of polyclonal, monoclonal or recombinant antibodies against the oligopeptide(s) concerned or against a complex of the oligopeptide(s) concerned and HLA-A2.

9. An antibody which reacts specifically with at least one oligopeptide as claimed in claim 1 and/or a retro-inverse peptide or pseudopeptide as claimed in claim 2 or with a complex of the oligopeptide(s) concerned and HLA-A2.

10. The oligopeptide as claimed in claim 1, characterized in that it is present in an association complex with MHC class I tetramers or pharmaceutically suitable carriers or other structures.

11. The retro-inverse peptide or pseudopeptide as claimed in claim 2, characterized in that it is present in an association complex with MHC class I tetramers or pharmaceutically suitable carriers or other structures.

12. The use of at least one oligopeptide as claimed in claim 1 and/or of a retro-inverse peptide or pseudopeptide as claimed in claim 2 or of a polynucleotide as claimed in claim 3 for the preparation of polyclonal or monoclonal or recombinant A2-restricted T-cell receptors or molecules functionally equivalent thereto against the oligopeptide(s) concerned.

13. A T-cell receptor or molecule functionally equivalent thereto, which reacts specifically with at least one oligopeptide as claimed in claim 1 and/or a retro-inverse peptide or pseudopeptide as claimed in claim 2.

14. A polynucleotide which codes for a T-cell receptor as claimed in claim 13.

15. An expression vector which possesses the ability to express a T-cell receptor as claimed in claim 13.
